SEQUENCE LISTING (43 sequences)

SEQ ID NO: 1 - protein, 183 aa, Hepatitis B virus
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 2 - protein, 183 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 3 - DNA, 504 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaagaaaaagaagctcgagcaccaccaccaccaccac

SEQ ID NO: 4 - protein, 168 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKKLEHHHHHH

SEQ ID NO: 5 - DNA, 510 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaagaagaaaaagaagaagctcgagcaccaccaccaccaccac

SEQ ID NO: 6 - protein, 170 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKKKKLEHHHHHH

SEQ ID NO: 7 - DNA, 516 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaaaaagaagaagaaaaagaagaagctcgagcaccaccaccaccaccac

SEQ ID NO: 8 - protein, 172 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKKKKKKLEHHHHHH

SEQ ID NO: 9 - DNA, 519 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaaaaagaagaagaagaagaagaagaaactcgagcaccaccaccaccaccac

SEQ ID NO: 10 - protein, 173 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKKKKKKKLEHHHHHH

SEQ ID NO: 11 - DNA, 522 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaagaagaaaaagaagaagaaaaagaagaagctcgagcaccaccaccaccaccac

SEQ ID NO: 12 - protein, 174 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKKKKKKKKLEHHHHHH

SEQ ID NO: 13 - DNA, 528 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaaaaagaagaagaaaaagaagaagaaaaagaagaagctcgagcaccaccaccaccaccac

SEQ ID NO: 14 - protein, 176 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKKKKKKKKKKLEHHHHHH

SEQ ID NO: 15 - DNA, 549 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaaaaagaagaagaagaagaagaagaaaaagaagaagaagaagaagaagaagaaaaagctcgagcaccaccaccaccaccac

SEQ ID NO: 16 - protein, 183 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKKKKKKKKKKKKKKKKKLEHHHHHH

SEQ ID NO: 17 - DNA, 28 bp, Artificial sequence
  Chemically synthesized primer
cgactcactataggggaattgtgagcgg

SEQ ID NO: 18 - DNA, 45 bp, Artificial sequence
  Chemically synthesized primer
ggcctcgagcttctttttcttctttgcggccgcaagcttgtcgac

SEQ ID NO: 19 - DNA, 51 bp, Artificial sequence
  Chemically synthesized primer
ggcctcgagcttcttctttttcttcttctttgcggccgcaagcttgtcgac

SEQ ID NO: 20 - DNA, 57 bp, Artificial sequence
  Chemically synthesized primer
ggcctcgagcttcttctttttcttcttctttttctttgcggccgcaagcttgtcgac

SEQ ID NO: 21 - DNA, 60 bp, Artificial sequence
  Chemically synthesized primer
ggcctcgagtttcttcttcttcttcttcttctttttctttgcggccgcaagcttgtcgac

SEQ ID NO: 22 - DNA, 63 bp, Artificial sequence
  Chemically synthesized primer
ggcctcgagcttcttctttttcttcttctttttcttcttctttgcggccgcaagcttgtcgac

SEQ ID NO: 23 - DNA, 69 bp, Artificial sequence
  Chemically synthesized primer
ggcctcgagcttcttctttttcttcttctttttcttcttctttttctttgcggccgcaagcttgtcgac

SEQ ID NO: 24 - DNA, 90 bp, Artificial sequence
  Chemically synthesized primer
ggcctcgagctttttcttcttcttcttcttcttcttctttttcttcttcttcttcttcttctttttctttgcggccgcaagcttgtcgac

SEQ ID NO: 25 - protein, 12 aa, Hepatitis B virus
GPGAPGLVPRGS

SEQ ID NO: 26 - protein, 12 aa, Hepatitis B virus
GPASGPGIEGRA

SEQ ID NO: 27 - protein, 23 aa, Hepatitis B virus
CARGARGARGARGILGVFILLYM

SEQ ID NO: 28 - DNA, 570 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtgcgtcgccgtggtcgcagcccgcgccgtcgtaccccgagcccgcgtcgtcgtcgtagccagagcccgcgtcgtcgccgcagccagagccgcgaaagccagctcgagcaccaccaccaccaccac

SEQ ID NO: 29 - protein, 190 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVRRRGRSPRRRTPSPRRRRSQSPRRRRSQSRESQLEHHHHHH

SEQ ID NO: 30 - DNA, 492 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgcgatccggcgagccgcgatctggttgtgaactatgtgaataccaacatgggcctgaaaattcgtctgctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtcgacaagcttgcggccgcactcgagcaccaccaccaccaccactga

SEQ ID NO: 31 - DNA, 42 bp, Artificial sequence
  si RNA
ggaaagacuguuccaaaaauuuuccuuucugacaagguuuuu

SEQ ID NO: 32 - DNA, 19 bp, Artificial sequence
  si RNA
gcugacccugaaguucauc

SEQ ID NO: 33 - DNA, 19 bp, Artificial sequence
  si RNA
cgacugggacuucaaguag

SEQ ID NO: 34 - DNA, 19 bp, Artificial sequence
  si RNA
gcugacccugaaguucauc

SEQ ID NO: 35 - DNA, 19 bp, Artificial sequence
  si RNA
cgacugggacuucaaguag

SEQ ID NO: 36 - DNA, 540 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaaggcaaaggcaaaggcgaaggcaaaggctaaggcgaaggctaaggcgaagctcgagcaccaccaccaccaccac

SEQ ID NO: 37 - protein, 180 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKAKAKAKAKAKAKAKAKLEHHHHHH

SEQ ID NO: 38 - DNA, 540 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagggtaagggcaagggtaagggcaagggtaagggcaagggcaagggtaagctcgagcaccaccaccaccaccac

SEQ ID NO: 39 - protein, 180 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKGKGKGKGKGKGKGKGKLEHHHHHH

SEQ ID NO: 40 - DNA, 528 bp, Hepatitis B virus
atggatatcgatccgtataaagaatttggcgccaccgtggaactgctgagctttctgccgagcgatttctttccgagcgtgcgtgatctgctggataccgcgagcgcgctgtatcgcgaagcgctggaaagcccggaacattgtagcccgcaccataccgccctgcgtcaggcgattctgtgctggggtgaactgatgaccctggcgacctgggttggcaacaacctgtgtgatccggcgagccgcgatctggttgtgaactatgtgaataccaacatgggcctgaaaattcgtcagctgctgtggtttcatatcagctgcctgacctttggccgcgaaaccgtgctggaatatctggtgagctttggcgtttggatccgtaccccgccggcgtatcgtccgccgaatgcgccgattctgagcaccctgccggaaaccaccgttgtggacaagcttgcggccgcaaagaaaaagaagagccagagcccgaagaagaagaagaaactcgagcaccaccaccaccaccac

SEQ ID NO: 41 - protein, 176 aa, Hepatitis B virus
MDIDPYKEFGATVELLSFLPSDFFPSVRDLLDTASALYREALESPEHCSPHHTALRQAILCWGELMTLATWVGNNLCDPASRDLVVNYVNTNMGLKIRQLLWFHISCLTFGRETVLEYLVSFGVWIRTPPAYRPPNAPILSTLPETTVVDKLAAAKKKKSQSPKKKKKLEHHHHHH

SEQ ID NO: 42 - DNA, 81 bp, Artificial sequence
  Chemically synthesized primer
ggcctcgagcttcgccttagccttcgccttagcctttgccttcgccttagcctttgcctttgcggccgcaagcttgtcgac

SEQ ID NO: 43 - DNA, 69 bp, Artificial sequence
  Chemically synthesized primer
ggcctcgagtttcttcttcttcttcgggctctggctcttctttttctttgcggccgcaagcttgtcgac

(19) United States
(12) Patent Application Publication
(10) Pub. No.: US 2014/0010885 A1
(43) Pub. Date: Jan. 9, 2014

(54) SELF-ASSEMBLING NANOPARTICLE DRUG DELIVERY SYSTEM

(71) Applicants: Miguel de los Rios, Del Mar, CA (US); Kenneth J. Oh, San Diego, CA (US); Timothy L. Bullock, Goleta, CA (US); Patrick T. Johnson, Del Mar, CA (US); Jacek Ostrowski, Encinitas, CA (US)

(72) Inventors: Miguel de los Rios, Del Mar, CA (US); Kenneth J. Oh, San Diego, CA (US); Timothy L. Bullock, Goleta, CA (US); Patrick T. Johnson, Del Mar, CA (US); Jacek Ostrowski, Encinitas, CA (US)

(21) Appl. No.: 13/353,222

(22) Filed: Jan. 18, 2012

Related U.S. Application Data
(63) Continuation of application No. 12/082,154, filed on Apr. 9, 2008, now abandoned.
(60) Provisional application No. 60/910,704, filed on Apr. 9, 2007.

(51) Int. Cl. A61K 9/51; A61K 9/50

(52) U.S. Cl. CPC A61K 9/5169; A61K 9/5089
USPC 424/491; 424/184.1; 424/130.1; 424/172.1; 514/44 R; 514/44 A; 514/1.1; 530/350; 536/23.72; 435/375; 264/4.3

(57) ABSTRACT

A self-assembling nanoparticle drug delivery system for the delivery of various bioactive agents including peptides, proteins, nucleic acids or synthetic chemical drugs is provided. The self-assembling nanoparticle drug delivery system described herein includes viral capsid proteins, such as Hepatitis B Virus core protein, encapsulating the bioactive agent, a lipid layer or lipid/cholesterol layer coat and targeting or facilitating molecules anchored in the lipid layer. A method for construction of the self-assembling nanoparticle drug delivery system is also provided.

### RELATED APPLICATIONS

This application claims priority to, and the benefit of, U.S. Patent Application No. 60/910,704, filed Apr. 9, 2007. The entire contents of this application is incorporated by reference in its entirety.

### FIELD OF THE INVENTION

The present invention relates to methods for drug or bioactive agent delivery. Specifically, the present invention relates to a self-assembling drug or bioactive agent delivery system comprised of a bioactive agent captured within viral capsid proteins and coated or encapsulated with a lipid layer.

### BACKGROUND OF THE INVENTION

The development of drug delivery systems for small molecules, proteins and DNA have been greatly influenced by nanotechnology. Improved drug delivery systems can address issues associated with currently used drugs such as increasing efficacy or improving safety and patient compliance (Rocco M C and Bainbridge W S, eds Social Implications of Nanoscience and Technology, National Science Foundation Report, 2001). In addition, this technology permits the delivery of drugs that are highly insoluble or unstable in biological environments.

There has been considerable research into developing biodegradable nanoparticles as effective drug delivery systems (Panyam J et al., Biodegradable nanoparticles for drug and gene delivery to cells and tissue, Adv Drug Deliv Rev. 55:329-47, 2003). Nanoparticles are solid, colloidal particles consisting of macromolecular substances that vary in size from 10-1000 nanometers. The drug of interest is either dissolved, entrapped, adsorbed, attached or encapsulated into the nanoparticle matrix. The nanoparticle matrix can be comprised of biodegradable materials such as polymers or proteins. Depending on the method of preparation, nanoparticles can be obtained with different properties and release characteristics for the encapsulated therapeutic agents (Sahoo S K and Labhasetwar V, Nanotech approaches to drug delivery and imaging, DDT 8:1112-1120, 2003).

Although nanoparticle drug delivery provides many advantages, such as their ability to penetrate cells due to their small size or there ability to permit sustained drug release within the target site over a period of days or even weeks, there is a need for improved nanoparticle compositions and systems capable delivering various therapeutically beneficial biological and chemical agents to a wide variety of tissues effectively and efficiently.

### SUMMARY OF THE INVENTION

The present invention provides a self-assembling nanoparticle drug delivery system comprising any viral capsid protein which self-assembles into a capsid from a single protein monomer that can exist as a monomer, dimmer or larger complex, a bioactive agent captured within the capsid; and a complex lipid mixture coating the capsid. Preferably, the capsid is comprised of altered, mutated or engineered HBV core proteins that can improve the binding affinity of the bioactive agent to the carboxyl terminal portion of the HBV core proteins within the capsid.

The present invention also provides methods for forming a self-assembling nanoparticle drug delivery system comprising mixing a bioactive agent with an HBV core protein in the presence of a chemical denaturant or denaturing agent at a concentration of about 1M to about 3M, preferably about 1.5M to about 2.5M, to form a cage solution; encapsulating the bioactive agent in the core protein cage by raising the ionic strength of the cage solution to obtain a final salt concentration of about 50 mM to about 600 mM and decreasing the chemical denaturant or denaturing agent concentration to about 0.5M to about 4M, preferably about 0.75M to about 2M; adding a lipid linker molecule to facilitate lipid coating of the core protein to the cage solution; adding a complex lipid coating material comprised of POPG, cholesterol, and HSPC at a mass value of about 10% to about 60% of total protein to the cage solution to form a nanoparticle, preferably about 20% to about 40%, more preferably about 25% to about 35%; and purifying the nanoparticles.

The present invention also provides methods of regulating gene expression in a cell comprising administering a self-assembling nanoparticle drug delivery system containing a captured bioactive agent, where the bioactive agent can be a therapeutic agent such as a drug, protein, peptide or nucleic acid. In one preferred embodiment, the bioactive agent is siRNA, where the siRNA interferes with the mRNA of the gene to be regulated, thereby regulating expression of the gene.

The present invention also provides various novel peptides and nucleic acid molecules comprising amino acids 1-149 of SEQ ID NO: 1 or 2 and further comprising poly-lysine and poly-histidine domains at the carboxyl terminal tail. The poly-lysine and poly-histidine domains add at least five consecutive lysine residues and at least six histidine residues to the carboxyl terminal tail. The lysine residues added to the carboxyl terminus increase the polypeptide binding affinity for siRNA (about 18 to about 27 nucleotides in length) to about 50 nM to about 500 nM, preferably about 50 nM to about 300 nM, more preferably about 100 nM to about 200 nM. The present invention also provides a nucleic acid molecule comprising the nucleic acid sequence of SEQ ID NOs: 3, 5, 7, 9, 11, 13, 15, 36, 38 or 40 and a polypeptide comprising the amino acid sequence of SEQ ID NOs: 4, 6, 7, 10, 12, 14, 16, 37, 39 or 41.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a computational reconstruction depicting wild-type Hepatitis B Virus (HBV) capsid reconstructed from electron density maps of the full size HBV dimer from the perspective of looking down at the 6-fold axis.

FIG. 2 is a schematic depicting phosphatidyl ethanolamine (PE) conjugation to protein cage via a succinimidyl-4-(p-maleimidophenyl)butyrate (SMPB) intermediate.

FIG. 3 is a schematic depicting PE conjugation to protein cage via m-maleimidobenzoyl-N-hydroxysuccinimide ester (MBS) intermediate.

FIG. 4 is a schematic depicting conjugating maleimide-containing intermediates to sulfhydryl-containing proteins.

FIG. 5 is a flow diagram depicting the construction of a self-assembling nanoparticle drug delivery system.

FIG. 6A is a photograph showing negative stained nanocage particles lacking a lipid layer (naked) at 200,000× magnification. FIG. 6B is a photograph showing lipid coated nanocages stained with 1% PTA at 200,000× magnification. FIG. 6C is a photograph showing lipid coated nanocages with surface attached anti-CD22 antibodies stained with 1% PTA at 200,000× magnification.

FIG. 7 is a photograph of a gel showing a gel shift assay to determine the ability of nanocages with various C-terminal to encapsulate RNA.

FIG. 8 is a bar graph showing the comparison of antibody targeted cage (anti-CD22 HSPC cage) and non-targeted cage (HSPC only) binding to mCD22Ig.

FIG. 9 is a bar graph comparing the binding to mCD22Ig of anti-CD22 targeted nanocages over that of non-targeted nanocages.

FIG. 10 is a bar graph showing two identical ELISA experiments demonstrating that significantly more anti-CD22 targeted nanocage binding to mCD22Ig than non-targeted nanocages.

FIG. 11 is a bar graph showing that anti-CD22 targeted nanocages bind to B Cells (Ramos cells) significantly better than non-targeted nanocages.

FIG. 12A is a line graph showing that anti-CD22 targeted nanocages bind to B cells (BCL1) with more specificity than they bind to T Cells (Jurkat). FIG. 12B is a photograph showing a bright-field view of semi-confluent BCL1 cells (sub panel a), showing nuclei following counter stained with Hoechst 33342 (sub panel b) and showing internalized nanocages within all cells at 3 nm (sub panel c).

FIG. 13A are photographs showing the concentration-dependent (100 nM and 2.5 nM) internalization of anti-CD22 targeted nanocages and non-targeted nanocages in BCL1 cells. FIG. 13B is a line graph showing the dose-response of anti-CD22 targeted nanocages and non-targeted nanocages in BCL1 cells.

FIG. 14 is a line graph showing that “free” anti-CD22 antibody containing preparations (pink) mixed with purified anti-CD22 targeted nanocages (yellow) results in a >100-fold shift in the dose-response relationship of nanocage internalization in B Cells.

FIG. 15 is a photograph of a gel showing the degradation of free RNA as compared to caged RNA.

FIG. 16 is a graphic representation of the gel photograph of FIG. 14.

FIG. 17 is a photograph of a gel showing serum stability of the free RNA, RNA mixed with empty lipid coated nanocages, and lipid coated nanocages loaded with RNA.

FIG. 18 is a graphic representation of the gel photograph of FIG. 16.

FIG. 19 is a photograph of a gel showing free, caged, and protein-bound RNA migrating separately.

FIG. 20 is a graphic representation of the gel photograph of FIG. 18.

FIG. 21 is a photograph of a gel showing a gel shift assay to determine the affinity of K7 and K11 mutant proteins for a small amount (10 nM) of fluorescent siRNA.

FIG. 22 is a line graph showing the binding curves for K7 and K11 mutants.

FIG. 23 is a series of photographs of fluorescent cell staining showing that lipid coated nanocages containing red fluorescent-labeled siRNA can enter C166-eGFP cells.

FIG. 24 is a bar graph showing the knock down eGFP mRNA expression using lipid coated nanocages containing siRNA directed against eGFP.

FIG. 25 is a series of photographs of fluorescent cell staining showing that lipid coated nanocages containing red fluorescent siRNA directed against eGFP enters cells and knocks down eGFP protein expression.

FIG. 26 is a series of photographs of fluorescent cell staining showing that lipid coated nanocages containing red fluorescent siRNA directed against eGFP knock down eGFP protein expression in the mouse liver in vivo.

FIG. 27 is a fluorescent excitation and emission spectra for liver extracts match the corresponding spectra for EGFP.

FIG. 28 is a bar graph showing that liver fluorescence values were normalized by the amount of protein and reported as μM Fluorescein equivalents per mg/mL protein.

### DETAILED DESCRIPTION OF THE INVENTION

The present invention provides a novel nanoparticle drug or bioactive agent delivery system that can transport a wide range of chemical, biological and therapeutic molecules into the circulatory system following administration. The nanoparticles of the present invention comprise building blocks re-engineered from natural proteins which self-assemble to form nanocages. During the assembly process, bioactive agents are captured by the specific chemistries of the inward facing surfaces of the cage-forming blocks by simple diffusion/concentration mechanics. Coulombic interactions, disulfide interactions and hydrogen bonding mechanisms can also be engineered by specific mutations at or near the carboxyl terminus to further capture of the bioactive agents. The assembled cage has special functionalities to guide the assembly of a coat, which is a self-assembling layer of anionic, neutral or cationic lipids which can be mixed with varying ratios of cholesterol. Peptides that facilitate membrane transduction can be integrated into the lipid layer coat to endow the system with the ability to pass through cell walls. Polyethylene glycol (PEG) of varying chain lengths can also be anchored into the membrane for the purpose of eluding the immune system and to fend off attacking degradative enzymes. This multilayered delivery system orchestrates a complex arrangement of biomolecules and is entirely self-assembling.

The synthetic non-viral capsule is composed of re-engineered biological molecules and enhanced with synthetic chemical components. Although this design is inspired by the natural behavior of viruses, and uses viral capsid proteins as the building blocks, this system is inactive and non-replicating. In addition, all of the proteins used to make the building blocks of the system were all re-engineered to exhibit desired characteristics by altering stabilities and removing or adding disulfide linkages. The building blocks are designed so that once the cage starts to disintegrate, they are degraded quickly so as to limit any potential immune response. A characteristic of this drug delivery system is its ability to create the building blocks of the cage with bioactive agents attached to every unit. Yet another important feature of this system is the use of the beneficial characteristics of a virus to deliver molecules that no virus could deliver, such as synthetic drugs, without pathogenic potential. The nanoparticle drug delivery system does not incorporate an attenuated virus, but just the capsid, a shell of proteins that form regular geometric shapes. The terms capsid, cage and nanocage are used interchangeably herein to refer to the self-assembled capsid of viral capsid proteins.

Any viral capsid protein which self-assembles into a capsid from a single protein monomer is suitable for use in the nanoparticle drug delivery system of the present invention. Non-limiting examples of self-assembling capsid proteins include human and duck Hepatitis B Virus core protein, Hepatitis C Virus core protein, Human Papilloma Virus type 6 L1 and L2 protein and cowpea chlorotic mottle virus coat protein. An exemplary protein for constructing the nanocage of the nanoparticle drug delivery system is Hepatitis B Virus (HBV) core protein (C-protein) (SEQ ID NO. 1), a protein that naturally self-assembles to form the protein capsid of the virus. Different strains of HBV have slight variations in the sequence of C-protein. Any strain of HBV C-protein can be utilized. Core protein was chosen not only because it self-assembles into a capsid, but also because it is the only necessary component to form a complete capsid.

HBV C-protein of SEQ ID NO:1 has an amino acid sequence 1 to 183 (NCBI Protein Database Accession Number BAD86623):

| (SEQ ID NO: 1) | |
|---|---|
| MET ASP ILE ASP PRO TYR LYS GLU PHE GLY ALA SER VAL GLU LEU | (15) |
| LEU SER PHE LEU PRO SER ASP PHE PHE PRO SER ILE ARG ASP LEU | (30) |
| LEU ASP THR ALA SER ALA LEU TYR ARG GLU ALA LEU GLU SER PRO | (45) |
| GLU HIS CYS SER PRO HIS HIS THR ALA LEU ARG GLN ALA ILE LEU | (60) |
| CYS TRP GLY GLU LEU MET ASN LEU ALA THR TRP VAL GLY SER ASN | (75) |
| LEU GLU ASP PRO ALA SER ARG GLU LEU VAL VAL SER TYR VAL ASN | (90) |
| VAL ASN MET GLY LEU LYS ILE ARG GLN LEU LEU TRP PHE HIS ILE | (105) |
| SER CYS LEU THR PHE GLY ARG GLU THR VAL LEU GLU TYR LEU VAL | (120) |
| SER PHE GLY VAL TRP ILE ARG THR PRO PRO ALA TYR ARG PRO PRO | (135) |
| ASN ALA PRO ILE LEU SER THR LEU PRO GLU THR THR VAL VAL ARG | (150) |
| ARG ARG GLY ARG SER PRO ARG ARG ARG THR PRO SER PRO ARG ARG | (165) |
| ARG ARG SER GLN SER PRO ARG ARG ARG ARG SER GLN SER ARG GLU | (180) |
| SER GLN CYS | (183) |

An alternative HBV C-protein of SEQ ID NO:2 has an amino acid sequence 1 to 183 (NCBI Protein Database Accession Number AY741795):

| (SEQ ID NO: 2) | |
|---|---|
| MET ASP ILE ASP PRO TYR LYS GLU PHE GLY ALA THR VAL GLU LEU | (15) |
| LEU SER PHE LEU PRO SER ASP PHE PHE PRO SER VAL ARG ASP LEU | (30) |
| LEU ASP THR ALA SER ALA LEU TYR ARG GLU ALA LEU GLU SER PRO | (45) |
| GLU HIS CYS SER PRO HIS HIS THR ALA LEU ARG GLN ALA ILE LEU | (60) |
| CYS TRP GLY GLU LEU MET THR LEU ALA THR TRP VAL GLY ASN ASN | (75) |
| LEU GLU ASP PRO ALA SER ARG ASP LEU VAL VAL ASN TYR VAL ASN | (90) |
| THR ASN MET GLY LEU LYS ILE ARG GLN LEU LEU TRP PHE HIS ILE | (105) |
| SER CYS LEU THR PHE GLY ARG GLU THR VAL LEU GLU TYR LEU VAL | (120) |
| SER PHE GLY VAL TRP ILE ARG THR PRO PRO ALA TYR ARG PRO PRO | (135) |
| ASN ALA PRO ILE LEU SER THR LEU PRO GLU THR THR VAL VAL ARG | (150) |
| ARG ARG GLY ARG SER PRO ARG ARG ARG THR PRO SER PRO ARG ARG | (165) |
| ARG ARG SER GLN SER PRO ARG ARG ARG ARG SER GLN SER ARG GLU | (180) |
| SER GLN CYS | (183) |

HBV C-protein assembles to form an icosahedral viral capsid. Viruses are macromolecular complexes, composed of a nucleic acid genome enclosed in a protein coat (or capsid) and sometimes a lipid membrane. Viral genomes are usually very small and can be composed of as few as three genes. The virus must, therefore, be extremely efficient in its use of genetic material and consequently the capsid (which protects the viral genome in the harsh extracellular environment) must assemble from a small number of gene products. Asymmetric viral protein monomers are arranged such that they occupy identical bonding environments. Spherical viruses, such as HBV, assemble as icosahedra, which are 20-sided polyhedra composed of 60 asymmetric unites arranged as equilateral triangles. The viral icosahedral capsids assemble from one protein species in 60ₙ subunits. These icosahedra are described by their triangulation number (T) where there are 60T subunits.

The full length HBV C-protein forms particles (T=4) with a diameter of approximately 36 nanometers (Crowther R A et al., Three-dimensional structure of hepatitis B virus core particles determined by electron cryomicroscopy, Cell 77:943-50, 1994). Inside this particle, the final 40 amino acids of the C-protein are thought to interact with the genomic DNA of the virus. Core protein constructs lacking this putative DNA-binding region also form icosahedral capsids, but with a triangulation number of three (T=3). Interactions between C-protein monomers in these two types of capsids are thought to be similar.

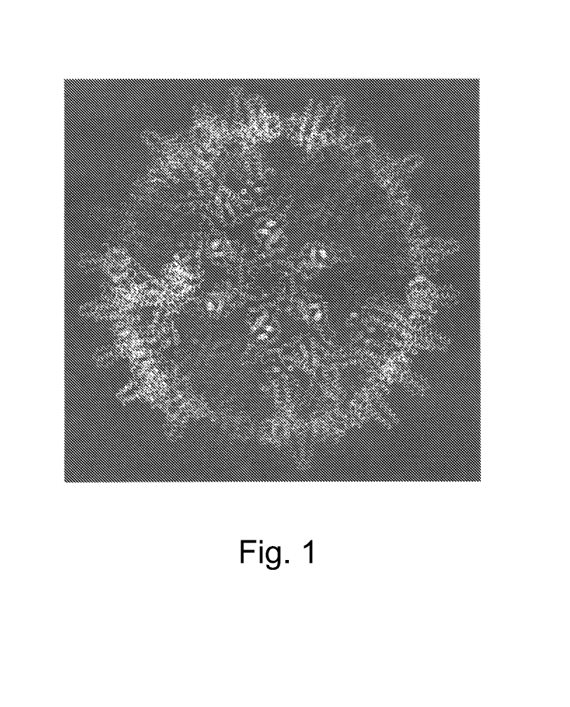

In HBV capsids, C-protein monomers form dimers that associate tightly via a “spike.” The spike is a central four alpha-helical bundle (Bottcher B et al., Determination of the fold of the C-protein of hepatitis B virus by electron cryomicroscopy, Nature 386:88-91, 1997) with a 2-fold axis of symmetry. The icosahedral viral capsid consists of 120 C-protein dimers assembled around 5-fold and 6-fold axes in a rough head-to-tail type interaction. In the mature virus, the tips of the central spikes of the 120 dimers are oriented close to the surface of the particle where it is coated by a plasma membrane. A computational reconstruction of wild-type HBV capsid reconstructed from electron density maps of the full size HBV dimmer with the perspective of looking down at the 6-fold axis is depicted in FIG. 1. FIG. 1 is representative of what a naked (comprised solely of capsid proteins) nanocage looks like prior to being coated with a lipid layer.

In vitro assembly of empty HBV capsids using the dimeric 149 residue assembly domain of the C-protein (amino acids 1-149) can be induced by increased ionic strength from about 50 mM to about 600 mM (e.g., high NaCl concentration). In HBV, subunit dimers are stable in solution. Assembly of HBV conforms to thermodynamic and kinetic predictions of the simplest case assembly models. Assembly reactions appear to contain only dimer and capsid and show a predicted steep concentration dependence. This assembly demonstrates a remarkably weak association constant, yet capsids assemble because subunits are multivalent. Capsids are even more stable than the association constant would predict because there is a steep energy barrier which inhibits disassociation (Zlotnick A, Are weak protein-protein interactions the general rule of capsid assembly? Virology 315:269-274, 2003).

In addition to the use of the naturally occurring HBV C-proteins (e.g., SEQ ID NO:1 and SEQ ID NO:2) in the nanoparticle drug delivery system, the present invention provides several modifications (e.g., alterations, truncations, mutations, etc.) to the C-protein sequences to enhance the structural and functional characteristics of the HBV C-proteins and provide superior nanoparticle drug delivery systems. These modifications to the HBV C-protein can be made, that is engineered, according to any method known in the art, including without limitation genetic engineering, chemical modification, etc. These modifications, inter alia: (a) strengthen and promote assembly of the HBV C-protein monomers into the capsid; (b) optimize binding and release of the desired bioactive agent captured within the capsid; (c) enhance and promote the coating of the capsid with a lipid layer or lipid/cholesterol layer, and/or (d) facilitate the disassembly of the entire capsid in the bloodstream following administration.

Capsid Assembly Modifications

Expressed C-protein in solution forms a dimer that is naturally stabilized by salt bridges, hydrophobic interactions, and covalent inter- and intra-molecular disulfide bonds. The intra-molecular bonds can be engineered so that C-protein stability can be tuned to a desired level. Additionally, inter-molecular disulfide bonds can be engineered so as to affect the stability of the cage. Specific salt bridges between dimers that help form the capsid can also be mutated to cysteines so that disulfide bonds form and stabilize the capsid structure.

In order to promote and strengthen the assembly of the HBV C-protein monomers into a nanocage capsid, modifications can be engineered into the HBV C-protein in the spike area of the dimer or the interface between dimers. These modifications can include the introduction of a pair of cysteines into this interface. For example, a first cysteine (e.g. amino acid 23) is introduced in the first position in order to form a disulfide bond with a second cysteine (amino acid 132 in this case) in a neighboring molecule. Similarly, the second position also participates in a disulfide bond, allowing the dimer to participate in four disulfide bridges and a total of 180 stabilizing covalent interactions. Four different types of disulfide bonds, according to their effectiveness in stabilizing the assembly and the desired strength of the assembly, can be created:

Mutation 1: Phenylalanine 23 to cysteine; tyrosine 132 to cysteine

Mutation 2: Aspartic acid 29 to cysteine; arginine 127 to cysteine

Mutation 3: Threonine 33 to cysteine; valine 124 to cysteine

Mutation 4: Leucine 37 to cysteine; valine 120 to cysteine

All modifications of C-protein are based on an extensive analysis of the capsid crystal structure and energy minimization models performed on electron density maps derived from structural data. Other modifications can be engineered based from this structural data.

Bioactive Agent Binding Modifications

The wild type HBV C protein is 183 amino acids of which the first 149 amino acids form a globular fold followed by a 35 amino acid C-terminal tail. Various modifications of the C-terminal tail can be engineered to provide the appropriate properties for binding the bioactive agent to the nanocage where the binding affinity of the C-terminal tail is at a sub-micromolar (or stronger) affinity for the bioactive agent.

The 35 amino C-terminal tail is presumed to hang inside the fully formed viral capsid and bind the viral nucleic acid. It consists of 4 arginine-rich repeats. This cluster of positive charges at the C-terminus can interact with negatively charged molecules such as DNA or RNA. The C-terminal tail can be truncated to various lengths to present one to four of these arginine-rich repeats or can be completely truncated to remove all four arginine-rich repeats. The C-protein can also be engineered so that the C-terminal tail has a cluster of negative charges (Asp or Glu residues) that can interact with positively charged molecules.

In preferred embodiments, the complete C-terminal tail can be truncated and a tail can be substituted which contains one or more poly-lysine domains, with c-terminal poly histidine-tags. The truncation mutations creating various poly-lysine domains of differing lengths after the first 149 amino acids of HBV core protein can be engineered using any methods known in the art. In one embodiment, the core protein gene can be amplified via PCR up to amino acid 149 and various numbers of lysine (or other) residues can be added to amino acids 1-149. A linker may be optionally present between the amino acid residue 149 and the domain that binds the bioactive agent that is added at the C-terminal tail. In some embodiments, the linker is about 3 amino acids to about 15 amino acids in length (or any specific amino acid length disposed with the range) and can link the poly-lysine domain to amino acid 149 of the HBV core protein and provide flexibility to the C-terminal tail. In some embodiments, the poly-lysine domain can be followed by a poly histidine tag and/or followed by an XhoI restriction site. The poly histidine tag can include at least six histidine residues added to the C-terminal tail. Modifications to the C-terminal tail can include the addition of one or more poly-lysine domains. When more than one poly-lysine domain is present, the poly-lysine domains can be separated by about 1 to about 20 amino acid residues (Each poly-lysine domain can comprise about one to about thirty lysine residues. In some embodiments the poly-lysine domain can comprise about 5 lysine residues to about 20 lysine residues. In some embodiments, where more than one poly-lysine domain is present the each poly-lysine domain can comprise about 4 lysine residues to about 20 lysine residues (or any specific amino acid length disposed with the range). In some embodiments, at least four or at least five consecutive lysine residues are added to the C-terminal tail. Poly-lysine domains and poly histidine tag can be added to the C-terminal tails separately or in combination. The poly histidine tag can be included in some embodiments to facilitate purification of the proteins. C-terminal tails of 5 lysines (K5), 7 lysines (K7), 9 lysines (K9), 10 lysines (K10), 11 lysines (K11), 13 lysines (K13), 20 lysines (K20) were constructed. Additional C-terminal tails were conducted including a poly-lysine region with nine lysines alternating with a poly-alanine region with nine alanines (KA9), a poly-lysine region with nine lysines alternating with a poly-glycine region with nine glycines (KG9) and a poly-lysine region with nine lysines interrupted by a sequence of at least four amino acids between the fourth and fifth lysines (K4-5). Preferably, the four amino acid stretch between the fourth and fifth lysines of the K4-5 tail can be amino acids Ser-Gln-Ser-Pro.

The addition of the poly-lysine domains can increase the binding affinity of negatively charged molecules such as DNA or RNA for the core protein. The poly-lysine domains can increase the binding affinity (e.g., tight affinity, characteristic of RNA-protein interactions) of single or double stranded RNA (e.g., iRNA, siRNA, shRNA) for the core protein to K_{d} of about 50 nM to about 400 nM, about 50 nM to about 300 nM, about 50 nM to about 200 nM or about 50 nM to about 100 nM, or any integer disposed within said ranges. Binding affinity can be determined by various methods known in the art such as surface plasmon resonance (SPR), radioactivity displacement, ELISA or gel shift assays, described herein. The single stranded or double stranded RNA (e.g., iRNA, siRNA, shRNA) captured within the core protein can be any length sufficient to provide a biological, chemical or therapeutic benefit. For example the single stranded or double stranded RNA can be from about 10 to about 30 nucleotides in length, about 15 to about 27 nucleotides in length, 18 to about 27 nucleotides in length, or any nucleotide length within such ranges. In preferred embodiments, the RNA can be 21 nucleotide length blunt end, 19 nucleotide length with a 2 nucleotide hangover or can be 27 nucleotide length blunt end. These binding affinity increases are described in Example 12.

K5 has the following nucleic acid sequence:

(SEQ ID NO: 3)
ATG GAT ATC GAT CCG TAT AAA GAA TTT GGC GCC ACC
GTG GAA CTG CTG AGC TTT CTG CCG AGC GAT TTC TTT
CCG AGC GTG CGT GAT CTG CTG GAT ACC GCG AGC GCG
CTG TAT CGC GAA GCG CTG GAA AGC CCG GAA CAT TGT
AGC CCG CAC CAT ACC GCC CTG CGT CAG GCG ATT CTG
TGC TGG GGT GAA CTG ATG ACC CTG GCG ACC TGG GTT
GGC AAC AAC CTG TGT GAT CCG GCG AGC CGC GAT CTG
GTT GTG AAC TAT GTG AAT ACC AAC ATG GGC CTG AAA
ATT CGT CAG CTG CTG TGG TTT CAT ATC AGC TGC CTG
ACC TTT GGC CGC GAA ACC GTG CTG GAA TAT CTG GTG
AGC TTT GGC GTT TGG ATC CGT ACC CCG CCG GCG TAT
CGT CCG CCG AAT GCG CCG ATT CTG AGC ACC CTG CCG
GAA ACC ACC GTT GTG GAC AAG CTT GCG GCC GCA AAG
AAG AAA AAG AAG CTC GAG CAC CAC CAC CAC CAC CAC

K5 has the following amino acid sequence:

(SEQ ID NO: 4)
MDIDPYKEFGATVELLSFLPSDFFPSVRDLLDTASALYREALESPEHCSP
HHTALRQAILCWGELMTLATWVGNNLCDPASRDLVVNYVNTNMGLKIRQL
LWFHISCLTFGRETVLEYLVSFGVWIRTPPAYRPPNAPILSTLPETTVVD
KLAAAKKKKKLEHHHHHH

K7 has the following nucleic acid sequence:

(SEQ ID NO: 5)
ATG GAT ATC GAT CCG TAT AAA GAA TTT GGC GCC ACC
GTG GAA CTG CTG AGC TTT CTG CCG AGC GAT TTC TTT
CCG AGC GTG CGT GAT CTG CTG GAT ACC GCG AGC GCG
CTG TAT CGC GAA GCG CTG GAA AGC CCG GAA CAT TGT
AGC CCG CAC CAT ACC GCC CTG CGT CAG GCG ATT CTG
TGC TGG GGT GAA CTG ATG ACC CTG GCG ACC TGG GTT
GGC AAC AAC CTG TGT GAT CCG GCG AGC CGC GAT CTG
GTT GTG AAC TAT GTG AAT ACC AAC ATG GGC CTG AAA
ATT CGT CAG CTG CTG TGG TTT CAT ATC AGC TGC CTG
ACC TTT GGC CGC GAA ACC GTG CTG GAA TAT CTG GTG
AGC TTT GGC GTT TGG ATC CGT ACC CCG CCG GCG TAT
CGT CCG CCG AAT GCG CCG ATT CTG AGC ACC CTG CCG
GAA ACC ACC GTT GTG GAC AAG CTT GCG GCC GCA AAG
AAG AAG AAA AAG AAG AAG CTC GAG CAC CAC CAC CAC
CAC CAC

K7 has the following amino acid sequence:

(SEQ ID NO: 6)
MDIDPYKEFGATVELLSFLPSDFFPSVRDLLDTASALYREALESPEHCSP
HHTALRQAILCWGELMTLATWVGNNLCDPASRDLVVNYVNTNMGLKIRQL
LWFHISCLTFGRETVLEYLVSFGVWIRTPPAYRPPNAPILSTLPETTVVD
KLAAAKKKKKKKLEHHHHHH

K9 has the following nucleic acid sequence:

(SEQ ID NO: 7)
ATG GAT ATC GAT CCG TAT AAA GAA TTT GGC GCC ACC
GTG GAA CTG CTG AGC TTT CTG CCG AGC GAT TTC TTT
CCG AGC GTG CGT GAT CTG CTG GAT ACC GCG AGC GCG
CTG TAT CGC GAA GCG CTG GAA AGC CCG GAA CAT TGT
AGC CCG CAC CAT ACC GCC CTG CGT CAG GCG ATT CTG
TGC TGG GGT GAA CTG ATG ACC CTG GCG ACC TGG GTT
GGC AAC AAC CTG TGT GAT CCG GCG AGC CGC GAT CTG
GTT GTG AAC TAT GTG AAT ACC AAC ATG GGC CTG AAA
ATT CGT CAG CTG CTG TGG TTT CAT ATC AGC TGC CTG
ACC TTT GGC CGC GAA ACC GTG CTG GAA TAT CTG GTG
AGC TTT GGC GTT TGG ATC CGT ACC CCG CCG GCG TAT
CGT CCG CCG AAT GCG CCG ATT CTG AGC ACC CTG CCG
GAA ACC ACC GTT GTG GAC AAG CTT GCG GCC GCA AAG
AAA AAG AAG AAG AAA AAG AAG AAG CTC GAG CAC CAC
CAC CAC CAC CAC

K9 has the following amino acid sequence:

(SEQ ID NO: 8)
MDIDPYKEFGATVELLSFLPSDFFPSVRDLLDTASALYREALESPEHCSP
HHTALRQAILCWGELMTLATWVGNNLCDPASRDLVVNYVNTNMGLKIRQL
LWFHISCLTFGRETVLEYLVSFGVWIRTPPAYRPPNAPILSTLPETTVVD
KLAAAKKKKKKKKKLEHHHHHH

K10 has the following nucleic acid sequence:

(SEQ ID NO: 9)
ATG GAT ATC GAT CCG TAT AAA GAA TTT GGC GCC ACC
GTG GAA CTG CTG AGC TTT CTG CCG AGC GAT TTC TTT
CCG AGC GTG CGT GAT CTG CTG GAT ACC GCG AGC GCG
CTG TAT CGC GAA GCG CTG GAA AGC CCG GAA CAT TGT
AGC CCG CAC CAT ACC GCC CTG CGT CAG GCG ATT CTG
TGC TGG GGT GAA CTG ATG ACC CTG GCG ACC TGG GTT
GGC AAC AAC CTG TGT GAT CCG GCG AGC CGC GAT CTG
GTT GTG AAC TAT GTG AAT ACC AAC ATG GGC CTG AAA
ATT CGT CAG CTG CTG TGG TTT CAT ATC AGC TGC CTG
ACC TTT GGC CGC GAA ACC GTG CTG GAA TAT CTG GTG
AGC TTT GGC GTT TGG ATC CGT ACC CCG CCG GCG TAT
CGT CCG CCG AAT GCG CCG ATT CTG AGC ACC CTG CCG
GAA ACC ACC GTT GTG GAC AAG CTT GCG GCC GCA AAG
AAA AAG AAG AAG AAG AAG AAG AAG AAA CTC GAG CAC
CAC CAC CAC CAC CAC

K10 has the following amino acid sequence:

(SEQ ID NO: 10)
MDIDPYKEFGATVELLSFLPSDFFPSVRDLLDTASALYREALESPEHCSP
HHTALRQAILCWGELMTLATWVGNNLCDPASRDLVVNYVNTNMGLKIRQL
LWFHISCLTFGRETVLEYLVSFGVWIRTPPAYRPPNAPILSTLPETTVVD
KLAAAKKKKKKKKKKLEHHHHHH

K11 has the following nucleic acid sequence:

(SEQ ID NO: 11)
ATG GAT ATC GAT CCG TAT AAA GAA TTT GGC GCC ACC
GTG GAA CTG CTG AGC TTT CTG CCG AGC GAT TTC TTT
CCG AGC GTG CGT GAT CTG CTG GAT ACC GCG AGC GCG
CTG TAT CGC GAA GCG CTG GAA AGC CCG GAA CAT TGT
AGC CCG CAC CAT ACC GCC CTG CGT CAG GCG ATT CTG
TGC TGG GGT GAA CTG ATG ACC CTG GCG ACC TGG GTT
GGC AAC AAC CTG TGT GAT CCG GCG AGC CGC GAT CTG
GTT GTG AAC TAT GTG AAT ACC AAC ATG GGC CTG AAA
ATT CGT CAG CTG CTG TGG TTT CAT ATC AGC TGC CTG
ACC TTT GGC CGC GAA ACC GTG CTG GAA TAT CTG GTG
AGC TTT GGC GTT TGG ATC CGT ACC CCG CCG GCG TAT
CGT CCG CCG AAT GCG CCG ATT CTG AGC ACC CTG CCG
GAA ACC ACC GTT GTG GAC AAG CTT GCG GCC GCA AAG
AAG AAG AAA AAG AAG AAG AAA AAG AAG AAG CTC GAG
CAC CAC CAC CAC CAC CAC

K11 has the following amino acid sequence:

(SEQ ID NO: 12)
MDIDPYKEFGATVELLSFLPSDFFPSVRDLLDTASALYREALESPEHCSP
HHTALRQAILCWGELMTLATWVGNNLCDPASRDLVVNYVNTNMGLKIRQL
LWFHISCLTFGRETVLEYLVSFGVWIRTPPAYRPPNAPILSTLPETTVVD
KLAAAKKKKKKKKKKKLEHHHHHH

K13 has the following nucleic acid sequence:

(SEQ ID NO: 13)
ATG GAT ATC GAT CCG TAT AAA GAA TTT GGC GCC ACC
GTG GAA CTG CTG AGC TTT CTG CCG AGC GAT TTC TTT
CCG AGC GTG CGT GAT CTG CTG GAT ACC GCG AGC GCG
CTG TAT CGC GAA GCG CTG GAA AGC CCG GAA CAT TGT
AGC CCG CAC CAT ACC GCC CTG CGT CAG GCG ATT CTG
TGC TGG GGT GAA CTG ATG ACC CTG GCG ACC TGG GTT
GGC AAC AAC CTG TGT GAT CCG GCG AGC CGC GAT CTG
GTT GTG AAC TAT GTG AAT ACC AAC ATG GGC CTG AAA
ATT CGT CAG CTG CTG TGG TTT CAT ATC AGC TGC CTG
ACC TTT GGC CGC GAA ACC GTG CTG GAA TAT CTG GTG
AGC TTT GGC GTT TGG ATC CGT ACC CCG CCG GCG TAT
CGT CCG CCG AAT GCG CCG ATT CTG AGC ACC CTG CCG
GAA ACC ACC GTT GTG GAC AAG CTT GCG GCC GCA AAG
AAA AAG AAG AAG AAA AAG AAG AAG AAA AAG AAG AAG
CTC GAG CAC CAC CAC CAC CAC CAC

K13 has the following amino acid sequence:

(SEQ ID NO: 14)
MDIDPYKEFGATVELLSFLPSDFFPSVRDLLDTASALYREALESPEHCSP
HHTALRQAILCWGELMTLATWVGNNLCDPASRDLVVNYVNTNMGLKIRQL
LWFHISCLTFGRETVLEYLVSFGVWIRTPPAYRPPNAPILSTLPETTVVD
KLAAAKKKKKKKKKKKKKLEHHHHHH

K20 has the following nucleic acid sequence:

(SEQ ID NO: 15)
ATG GAT ATC GAT CCG TAT AAA GAA TTT GGC GCC ACC
GTG GAA CTG CTG AGC TTT CTG CCG AGC GAT TTC TTT
CCG AGC GTG CGT GAT CTG CTG GAT ACC GCG AGC GCG
CTG TAT CGC GAA GCG CTG GAA AGC CCG GAA CAT TGT
AGC CCG CAC CAT ACC GCC CTG CGT CAG GCG ATT CTG
TGC TGG GGT GAA CTG ATG ACC CTG GCG ACC TGG GTT
GGC AAC AAC CTG TGT GAT CCG GCG AGC CGC GAT CTG
GTT GTG AAC TAT GTG AAT ACC AAC ATG GGC CTG AAA
ATT CGT CAG CTG CTG TGG TTT CAT ATC AGC TGC CTG
ACC TTT GGC CGC GAA ACC GTG CTG GAA TAT CTG GTG
AGC TTT GGC GTT TGG ATC CGT ACC CCG CCG GCG TAT
CGT CCG CCG AAT GCG CCG ATT CTG AGC ACC CTG CCG
GAA ACC ACC GTT GTG GAC AAG CTT GCG GCC GCA AAG
AAA AAG AAG AAG AAG AAG AAG AAG AAA AAG AAG AAG
AAG AAG AAG AAG AAG AAA AAG CTC GAG CAC CAC CAC
CAC CAC CAC

K20 has the following amino acid sequence:

(SEQ ID NO: 16)
MDIDPYKEFGATVELLSFLPSDFFPSVRDLLDTASALYREALESPEHCSP
HHTALRQAILCWGELMTLATWVGNNLCDPASRDLVVNYVNTNMGLKIRQL
LWFHISCLTFGRETVLEYLVSFGVWIRTPPAYRPPNAPILSTLPETTVVD
KLAAAKKKKKKKKKKKKKKKKKKKKLEHHHHHH

KA9 has the following nucleic acid sequence:

(SEQ ID NO: 36)
ATG GAT ATC GAT CCG TAT AAA GAA TTT GGC GCC ACC GTG GAA CTG CTG AGC TTT
CTG CCG AGC GAT TTC TTT CCG AGC GTG CGT GAT CTG CTG GAT ACC GCG AGC GCG
CTG TAT CGC GAA GCG CTG GAA AGC CCG GAA CAT TGT AGC CCG CAC CAT ACC GCC
CTG CGT CAG GCG ATT CTG TGC TGG GGT GAA CTG ATG ACC CTG GCG ACC TGG GTT
GGC AAC AAC CTG TGT GAT CCG GCG AGC CGC GAT CTG GTT GTG AAC TAT GTG AAT
ACC AAC ATG GGC CTG AAA ATT CGT CAG CTG CTG TGG TTT CAT ATC AGC TGC CTG
ACC TTT GGC CGC GAA ACC GTG CTG GAA TAT CTG GTG AGC TTT GGC GTT TGG ATC
CGT ACC CCG CCG GCG TAT CGT CCG CCG AAT GCG CCG ATT CTG AGC ACC CTG CCG
GAA ACC ACC GTT GTG GAC AAG CTT GCG GCC GCA AAG GCA AAG GCA AAG GCG AAG
GCA AAG GCT AAG GCG AAG GCT AAG GCG AAG CTC GAG CAC CAC CAC CAC CAC CAC

KA9 has the following amino acid sequence:

(SEQ ID NO: 37)
MDIDPYKEFGATVELLSFLPSDFFPSVRDLLDTASALYREALESPEHCSPHHTALRQAILCWGELMTLATWVGN
NLCDPASRDLVVNYVNTNMGLKIRQLLWFHISCLTFGRETVLEYLVSFGVWIRTPPAYRPPNAPILSTLPETTV
VDKLAAAKAKAKAKAKAKAKAKAKLEHHHHHH

KG9 has the following nucleic acid sequence:

(SEQ ID NO: 38)
ATG GAT ATC GAT CCG TAT AAA GAA TTT GGC GCC ACC GTG GAA CTG CTG AGC TTT
CTG CCG AGC GAT TTC TTT CCG AGC GTG CGT GAT CTG CTG GAT ACC GCG AGC GCG
CTG TAT CGC GAA GCG CTG GAA AGC CCG GAA CAT TGT AGC CCG CAC CAT ACC GCC
CTG CGT CAG GCG ATT CTG TGC TGG GGT GAA CTG ATG ACC CTG GCG ACC TGG GTT
GGC AAC AAC CTG TGT GAT CCG GCG AGC CGC GAT CTG GTT GTG AAC TAT GTG AAT
ACC AAC ATG GGC CTG AAA ATT CGT CAG CTG CTG TGG TTT CAT ATC AGC TGC CTG
ACC TTT GGC CGC GAA ACC GTG CTG GAA TAT CTG GTG AGC TTT GGC GTT TGG ATC
CGT ACC CCG CCG GCG TAT CGT CCG CCG AAT GCG CCG ATT CTG AGC ACC CTG CCG
GAA ACC ACC GTT GTG GAC AAG CTT GCG GCC GCA AAG GGT AAG GGC AAG GGT AAG
GGC AAG GGT AAG GGC AAG GGC AAG GGT AAG CTC GAG CAC CAC CAC CAC CAC CAC

KG9 has the following amino acid sequence:

(SEQ ID NO: 39)
MDIDPYKEFGATVELLSFLPSDFFPSVRDLLDTASALYREALESPEHCSPHHTALRQAILCWGELMTLATWVGN
NLCDPASRDLVVNYVNTNMGLKIRQLLWFHISCLTFGRETVLEYLVSFGVWIRTPPAYRPPNAPILSTLPETTV
VDKLAAAKGKGKGKGKGKGKGKGKLEHHHHHH

K4-5 has the following nucleic acid sequence:

(SEQ ID NO: 40)
ATG GAT ATC GAT CCG TAT AAA GAA TTT GGC GCC ACC GTG GAA CTG CTG AGC TTT
CTG CCG AGC GAT TTC TTT CCG AGC GTG CGT GAT CTG CTG GAT ACC GCG AGC GCG
CTG TAT CGC GAA GCG CTG GAA AGC CCG GAA CAT TGT AGC CCG CAC CAT ACC GCC
CTG CGT CAG GCG ATT CTG TGC TGG GGT GAA CTG ATG ACC CTG GCG ACC TGG GTT
GGC AAC AAC CTG TGT GAT CCG GCG AGC CGC GAT CTG GTT GTG AAC TAT GTG AAT
ACC AAC ATG GGC CTG AAA ATT CGT CAG CTG CTG TGG TTT CAT ATC AGC TGC CTG
ACC TTT GGC CGC GAA ACC GTG CTG GAA TAT CTG GTG AGC TTT GGC GTT TGG ATC
CGT ACC CCG CCG GCG TAT CGT CCG CCG AAT GCG CCG ATT CTG AGC ACC CTG CCG
GAA ACC ACC GTT GTG GAC AAG CTT GCG GCC GCA AAG AAA AAG AAG AGC CAG AGC
CCG AAG AAG AAG AAG AAA CTC GAG CAC CAC CAC CAC CAC CAC

K4-5 has the following amino acid sequence:

(SEQ ID NO: 41)
MDIDPYKEFGATVELLSFLPSDFFPSVRDLLDTASALYREALESPEHCSPHHTALRQAILCWGELMTLATWVGN
NLCDPASRDLVVNYVNTNMGLKIRQLLWFHISCLTFGRETVLEYLVSFGVWIRTPPAYRPPNAPILSTLPETTV
VDKLAAAKKKKSQSPKKKKKLEHHHHHH

The complete primer sequences, and peptide and nucleotide sequences used for the C-terminal tail constructs are described in Table 1.

TABLE 1
Primer Sequences

| Tail | | |
|---|---|---|
| Mutant | Forward Primer (5′ → 3′) | Reverse Primer (5′ → 3′) |
| K5 | CGACTCACTATAGGGGAATTGTGA | GGCCTCGAGCTTCTTTTTCTTCTTTGCGG |
| | GCGG (SEQ ID NO: 17) | CCGCAAGCTTGTCGAC (SEQ ID NO: 18) |
| K7 | CGACTCACTATAGGGGAATTGTGA | GGCCTCGAGCTTCTTCTTTTTCTTCTTCT |
| | GCGG (SEQ ID NO: 17) | TTGCGGCCGCAAGCTTGTCGAC (SEQ ID |
| | | NO: 19) |
| K9 | CGACTCACTATAGGGGAATTGTGA | GGCCTCGAGCTTCTTCTTTTTCTTCTTCT |
| | GCGG (SEQ ID NO: 17) | TTTTCTTTGCGGCCGCAAGCTTGTCGAC |
| | | (SEQ ID NO: 20) |
| K10 | CGACTCACTATAGGGGAATTGTGA | GGCCTCGAGTTTCTTCTTCTTCTTCTTCT |
| | GCGG (SEQ ID NO: 17) | TCTTTTTCTTTGCGGCCGCAAGCTTGTCG |
| | | AC (SEQ ID NO: 21) |
| K11 | CGACTCACTATAGGGGAATTGTGA | GGCCTCGAGCTTCTTCTTTTTCTTCTTCT |
| | GCGG (SEQ ID NO: 17) | TTTTCTTCTTCTTTGCGGCCGCAAGCTTG |
| | | TCGAC (SEQ ID NO: 22) |
| K13 | CGACTCACTATAGGGGAATTGTGA | GGCCTCGAGCTTCTTCTTTTTCTTCTTCT |
| | GCGG (SEQ ID NO: 17) | TTTTCTTCTTCTTTTTCTTTGCGGCCGCA |
| | | AGCTTGTCGAC (SEQ ID NO: 23) |
| K20 | CGACTCACTATAGGGGAATTGTGA | GGCCTCGAGCTTTTTCTTCTTCTTCTTCT |
| | GCGG (SEQ ID NO: 17) | TCTTCTTCTTTTTCTTCTTCTTCTTCTTCT |
| | | TCTTTTTCTTTGCGGCCGCAAGCTTGTCG |
| | | AC (SEQ ID NO: 24) |
| KA9 | CGACTCACTATAGGGGAATTGTGA | GGCCTCGAGCTTCGCCTTAGCCTTCGCC |
| | GCGG (SEQ ID NO: 17) | TTAGCCTTTGCCTTCGCCTTAGCCTTTGC |
| | | CTTTGCGGCCGCAAGCTTGTCGAC (SEQ |
| | | ID NO: 42) |
| KG9 | CGACTCACTATAGGGGAATTGTGA | GGCCTCGAGCTTCGCCTTAGCCTTCGCC |
| | GCGG (SEQ ID NO: 17) | TTAGCCTTTGCCTTCGCCTTAGCCTTTGC |
| | | CTTTGCGGCCGCAAGCTTGTCGAC (SEQ |
| | | ID NO: 42) |
| K4-5 | CGACTCACTATAGGGGAATTGTGA | GGCCTCGAGTTTCTTCTTCTTCTTCGGGC |
| | GCGG (SEQ ID NO: 17) | TCTGGCTCTTCTTTTTCTTTGCGGCCGCA |
| | | AGCTTGTCGAC (SEQ ID NO: 43) |

Although specific HBV core protein sequences with modified C-terminal sequences are disclosed, the invention is not limited to this specific sequences. One of skill in the art would recognize that nucleic acid and amino acid sequences about 75% to about 99% identical, about 80% to about 95% identical, about 85% to about 90% identical, or about 95% to about 99% identical, or any specific percent identity disposed within these ranges, to the nucleic acid sequences of SEQ ID NOs: 3, 5, 7, 9, 11, 13, 15, 36, 38 or 40 and the amino acid sequences of SEQ ID NOs: 4, 6, 7, 10, 12, 14, 16, 37, 39 or 41, which are capable of forming a nanocage and capable of binding and encapsulating a bioactive molecule are within the scope of the present invention.

The C-terminal tail of the C-protein can be replaced with a bioactive agent. The C-terminus can be engineered at the genetic level so as to create a chimeric building block of C-protein and the bioactive agent. The bioactive agent can be linked to the C-protein by a tether of amino acids that codes for a specific protease recognition site that permits the bioactive agent to be released once the nanocage begins to disassemble. The bioactive agent can also be linked to the C-protein though a disulfide bridge between cysteine residues in the C-terminal tail of C-protein and the agent. The cysteine residues can be those already present or they can be engineered at the desired location. The C-terminal tail can also be truncated to affect the natural association of molecules with the arginine rich tail or it can be exchanged with other known nucleic acid binding domains as described.

Capsid Disassembly Modifications

In order to facilitate the breakdown of the entire capsid, various alterations or mutations are made in the outer surface of the capsid to introduce blood protease recognition sequences. That is, once an HBV C-protein-derived nanoparticle has traveled into the bloodstream, it is necessary for it to disassemble into its component monomers so that it can release the encapsulated bioactive agent. To expedite this process, the HBV C-protein can be engineered so as to contain protease recognition sites at hinge and loop regions. The immunodominant spike of the C-protein can accommodate insertions of at least 46 residues and still be able to form capsids. The protease recognizes and cleaves this loop and thereby promotes disassembly. The two most commonly used blood proteases for this type of application are thrombin and factor Xa (Jenny R J et al., A critical review of the methods for cleavage of fusion proteins with thrombin and factor Xa, Protein Expr Purif 31:1-11, 2003). The specificities of these two proteases are well-known (Stevens R C, Drug Discovery World, 4:35-48, 2003) and can be readily incorporated into the internal loop of the C-protein. Thrombin is probably the best choice for specificity of these sites as there is known to be a constant, resting level of thrombin in the blood (Fernandez J A et al., Activated protein C correlates inversely with thrombin levels in resting healthy individuals, Am J. Hematol. 56:29-31, 1997). Sequences identified as SEQ ID NO. 25 and SEQ ID NO. 26 have a 12 amino acid extended loop and a recognition sequence for either thrombin:

(SEQ ID NO. 25)
GLY PRO GLY ALA PRO GLY LEU VAL PRO ARG GLY SER
or factor Xa
(SEQ ID NO. 26)
GLY PRO ALA SER GLY PRO GLY ILE GLU GLY ARG ALA

These sequences can be inserted into the spike region of the HBV C-protein (replacing amino acids 79 and 80 with these 12 amino insertion loops). These recognition sites add the benefit of quick degradation of the building blocks after the entire system has started to disassemble as a time-release method of distributing the encapsulated bioactive agents. This can minimize an immune response to the presence of “naked” C-protein in the blood stream.

Capsid Coating Modifications

In order to promote coating of the capsid by a lipid layer or lipid/cholesterol layer, various alterations or mutations are made in the outer surface of the capsid to introduce functional groups. In order to attach functional groups, either of the amino acids cysteine or lysine are placed at the tip of the spike in such a way as they protrude away from the capsid surface toward the plasma membrane. These modifications can permit the addition of one or more lipid linker molecules which can serve to promote or facilitate the lipid or lipid/cholesterol coat. For example, three positions (77, glutamic acid to cysteine; 78, aspartic acid to cysteine; and 80, alanine to cysteine) have been identified for the introduction of these amino acids which are functionalized at a later stage. Cysteine mutations can also be introduced at other locations in the C-protein. The choice of lysine or cysteine at each position is dependent of the orientation and geometry of each amino acid as judged from the crystal structure of the HBV capsid (Wynne S A et al., The crystal structure of the human hepatitis B virus capsid, Molecular Cell 3:771-80, 1999). Because of the 2-fold symmetry of the 4-helical bundle, an introduction of one reactive amino acid at each single position gives a total of two bioconjugated molecules per spike.

In a one embodiment, cysteine residues are engineered into the outer spike region of the capsid to provide a cross linker or activated lipid to bind the lipid layer to the protein nanocage. The cross linker or activated lipid can be any homo- or hetero-bifunctional linker known in the art. In a preferred embodiment, the activated lipid is phosphoethanolamine-malimide (PE-malimide or PE-mal).

In another embodiment, cysteine residues are engineered in the outer spike region of the capsid so that a modified Hepatitis B Virus S-protein can be covalently linked. The S-protein functions to guide the coating of the lipid layer or lipid/cholesterol layer. The S-proteins can be modified to have cysteines as well to complement the disulfide bridge formation between C-protein monomers.

Alternatively, the S-protein can be replaced by a peptide with similar characteristics to guide coating of the cage, such as a transmembrane engineered peptide. An exemplary transmembrane engineered peptide suitable for this purpose would have a flexible region that ends with a cysteine so as to form disulfide bridges with the cage. The opposite end of the peptide is comprised primarily of hydrophobic residues. A non-limiting example of such a HBV S-protein transmembrane engineered peptide has the amino acid sequence:

(SEQ ID NO: 27)
CYS ALA ARG GLY ALA ARG GLY ALA ARG GLY ALA ARG GLY ILE LEU (15)
GLY VAL PHE ILE LEU LEU TYR MET (23)

The hydrophobic region of this peptide associates with the hydrophobic lipid layer region, thus acting to guide the formation of a tight vesicle around the cage. These guiding peptides are added to the reaction mix after the formation of the cage and disulfide link to the C-protein.

In addition to the S-protein or equivalent transmembrane engineered peptides described above, phospholipids can be directly linked to the C-protein core to guide coating. At the apex of the spike region of core protein a cysteine residue is mutated as disclosed above and at this site fatty acids, including, but not limited to, modified phosphatidyl serine, are covalently attached. These fatty acids act as a guide for other phospholipids and cholesterols to coat the nanocage and form a layer around the nanocage. This replaces the necessity of an S-protein or a transmembrane engineered peptide. Also with the addition of these covalently attached phospholipids to the spike region (also known as the immunodominant spike), immune responses can be repressed.

The lipid layer can comprise phospholipids. Phospholipids suitable for forming the nanoparticle coat include, but are not limited to, hydrogenated soy phosphatidylcholine (HSPC), egg phosphatidylcholine (EPC), phosphatidyl ethanolamine (PE), phosphatidyl glycerol (PG), phosphatidyl inositol (PI), monosialogangolioside, spingomyelin (SPM), distearoylphosphatidylcholine (DSPC), dimyristoylphosphatidylcholine (DMPC), or dimyristoylphosphatidylglycerol (DMPG).

The lipid layer can partially or completely coat (or envelope) the protein nanocage. Preferably, the lipid layer completely coats the protein nanocage.

The lipid layer can be a lipid mono-layer, bi-layer or multi-laminar (or any combination thereof). The lipid layer can be attached to the protein nanocage by any suitable method in the art. Preferably, the lipid layer is covalently attached to the protein nanocage. In other preferred embodiments, the lipid layer is covalently attached to engineered locations in the protein nanocage (e.g., position 77, 78 or 80).

The coating components can further include 1-Palmitoyl-2-Oleoyl-sn-Glycero-3-[Phospho-rac-(1-glycerol)] (POPG).

The coating components can further include cholesterol, including a PEG-phospholipid. The PEG-phospholipid can comprise poly(ethylene glycol)-derivatized distearoylphosphatidylethanolamine (PEG-DSPE) and/or poly(ethylene glycol)-derivatized ceramides (PEG-CER).

The complex lipid coating material can be comprised of various amounts of cholesterol, HSPC or POPG. The lipid composite material can be about 5% to about 40% cholesterol, about 10% to about 80% HSPC and/or about 5% to about 80% POPG, or any specific percentage within said ranges. In some embodiments, the complex lipid coating material can be composed of: (a) 20% Cholesterol and 80% HSPC; (b) 50% Cholesterol and 50% HSPC; (c) 20% Cholesterol and 20% HSPC and 60% POPG; (d) 50% Cholesterol and 50% POPG; (e) 20% Cholesterol and 80% POPG; or (f) 10% Cholesterol and 15 HSPC and 65% POPG. Preferably, the lipid composite material is 20% Cholesterol and 20% HSPC and 60% POPG.

The complex lipid coating mixture can coat the nanocage at a mass value of about 10% to about 60%, about 10% to about 50%, about 15 to about 40%, about 20% to about 35% of the total protein (w/w), or any specific percentage with the recited ranges. The complex lipid coating mixture can coat the nanocage at a mass value of about 30% (w/w).

The nanoparticle coat can also be modified to allow the particles to evade the immune system and to enter the target cells. Cholesterol-tagged or lipid-tagged polyethylene glycol (PEG) and/or protein transduction domains (PTD) are added to the mixture. Non-limiting examples of suitable PTDs are the Human Immunodeficiency Virus (HIV) transactivator of transcription (Tat) peptide or poly-arginine (poly-Arg). First cholesterol-tagged PEG is anchored into the lipid layer and then cholesterol tagged PTDs are anchored into the lipid layer. The modified PEG and PTDs are added to the coated nanocages and insert into the coated surface in a concentration dependent manner.

Targeting Agents

Various targeting agents can be incorporated into the lipid layer or lipid/cholesterol layer coat to direct the nanoparticle to a tissue or cell target. In one example, the targeting agent is an antibody. Antibodies are comprised of two heavy and two light chains associated through disulfide bonds into two heavy chain-light chain complexes associated through exposed disulfide bonds in the heavy chain. In the presence of weak reducing agents such as β-mercaptoethanol, the heavy chains are dissociated leaving the heavy chain-light chain associations intact. Exposed sulfhydryl groups on the heavy chain can then be used to link the antibody to the free sulfate groups on the lipid coat. The resultant nanoparticles are comprised of drug encapsulated in a protein cages which are coated by lipid-targeting antibodies.

The lipids can be attached to antibodies through chemical means, such as reacting activated lipids such as PE-malimide to activated free amines of an antibody with agents such as Traut's Reagent. Lipid conjugated antibodies can then be incorporated into the lipid coat of the self-assembling nanoparticle drug delivery system.

The reduced antibody heavy chain-light chain complex above can also be attached directly to the naked protein cage. The protein building blocks can be engineered to incorporate cysteine residues with reactive sulfhydryl groups which then can be linked with the partially disassociated antibody chains. This configuration of nanoparticles results in drug encapsulated in a protein cage tagged with antibody targeting molecules.

Antibodies suitable for use as targeting agents in the nanoparticle drug delivery system include antibodies directed to cell surface antigens which cause the antibody-nanoparticle complex to be internalized, either directly or indirectly. Specific non-limiting examples of suitable antibodies include antibodies to CD19, CD20, CD22, CD33 and CD74. CD33 and CD22 are over-expressed and dimerized on lymphomas and binding to these antigens caused endocytosis and thereby internalization of the antibody-nanoparticle complex. Methods for incorporating incorporation of monoclonal antibodies to CD22 into the lipid coating can be found in U.S. Patent Publication No. 20070269370.

Bioactive Agents

The nanoparticle drug delivery system can be used to delivery a variety of therapeutically beneficial chemical compounds, bioactive agents and/or drugs. The terms chemical compounds, bioactive agents and drugs are used interchangeably herein. The individual nanoparticle of the nanoparticle drug delivery system can include one or more chemical compounds, bioactive agents and/or drugs.

The bioactive agents can include nucleic acids, DNA, RNA, siRNA, miRNA, shRNA, aptamers, antisense molecules, ribozymes, DNA vaccines, chemical compounds, small molecule chemical compounds, synthetically modified nucleic acid molecules, peptide nucleic acids (PNAs), peptides, nucleic acid mimetic molecules, peptides, polypeptides, peptidomimetics, carbohydrates, lipids or other organic or inorganic molecules. The term small molecule as used herein, is meant to refer to a composition that has a molecular weight of less than about 10 kD and most preferably less than about 5 kD.

Examples of bioactive agents suitable for use with the nanoparticle drug delivery system include, but are not limited to, cardiovascular drugs, respiratory drugs, cytotoxic agents sympathomimetic drugs, cholinomimetic drugs, adrenergic or adrenergic neuron blocking drugs, analgesics/antipyretics, anesthetics, antiasthmatics, antibiotics, antidepressants, antidiabetics, antifungals, antihypertensives, anti-inflammatories, antineoplastics, antianxiety agents, immunosuppressive agents, immunomodulatory agents, antimigraine agents, sedatives/hypnotics, antianginal agents, antipsychotics, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterials, antivirals, antimicrobials, anti-infectives, bronchodialators, hormones, hypoglycemic agents, hypolipidemic agents, proteins, peptides, nucleic acids, agents useful for erythropoiesis stimulation, antiulcer/antireflux agents, antinauseants/antiemetics and oil-soluble vitamins, or combinations thereof. The bioactive agent can be doxorubicin.

Expression of HBV C-Protein

The recombinant C-protein can expressed and purified using common molecular biology and biochemistry techniques. Recombinant expression vectors can be used which are engineered to carry the HBV C-protein gene into a host cell to provide for expression of the HBV C-protein. Such vectors can be introduced into a host cell by transfection means including, but not limited to, heat shock, calcium phosphate, DEAE-dextran, electroporation or liposome-mediated transfer. Recombinant expression vectors include, but are not limited to, Escherichia coli based expression vectors such as BL21 (DE3) pLysS, COS cell-based expression vectors such as CDM8 or pDC201, or CHO cell-based expression vectors such as pED vectors. The C-protein gene coding region can be linked to one of any number of promoters in an expression vector that can be activated in the chosen cell line. Additionally this cassette (capsid gene and promoter) is carried by a vector that contains a selectable marker such that cells receiving the vector can be identified.

Promoters to express the capsid proteins within a cell line can be drawn from those that are functionally active within the host cell. They can include, but are not limited to, the T7 promoter, the CMV promoter, the SV40 early promoter, the herpes TK promoter, and others well known in recombinant DNA technology. Inducible promoters can be used, including but not limited to, the metallothionine promoter (MT), the mouse mammary tumor virus promoter (MMTV), and others known to those skilled in the art.

Selectable markers and their attendant selection agents can be drawn from the group including, but not limited to, ampicillin, aminoglycoside phosphotransferase/G418, hygromycin-B phosphotransferase/hygromycin-B, and amplifiable selection markers such as dihydrofolate reductase/methotrexate and others known to skilled practitioners.

Eukaryotic, prokaryotic, insect, plant, and yeast expression systems can be utilized to express the HBV C-protein. In order to express capsid proteins the nucleotide sequence coding for the protein is inserted into an appropriate expression vector, i.e., a vector which contains the necessary elements for the transcription and translation of the inserted coding sequences. Methods which are well known to those skilled in the art can be used to construct expression vectors containing the protein coding sequences operatively associated with appropriate transcriptional/translational control signals. These methods include in vitro recombinant DNA techniques, synthetic techniques, and in vivo recombination/genetic recombination. See, for example, the techniques and vectors described in Maniatis, et al., 1989, Molecular Cloning, A Laboratory Manual, Cold Spring Harbor Laboratory, N.Y. and Ausubel et al., 1989, Current Protocols in Molecular Biology, Greene Publishing Associates & Wiley Interscience, N.Y.

A variety of eukaryotic, prokaryotic, insect, plant and yeast expression vector systems (e.g., vectors which contain the necessary elements for directing the replication, transcription, and translation of capsid protein coding sequences) can be utilized equally well by those skilled in the art, to express capsid protein coding sequences. These include but are not limited to microorganisms such as bacteria transformed with recombinant bacteriophage DNA, plasmid DNA or cosmid DNA expression vectors containing the capsid protein coding sequences; yeast transformed with recombinant yeast expression vectors containing the capsid protein coding sequences; insect cell systems infected with recombinant virus expression vectors (e.g., baculovirus) containing the capsid protein coding sequences; plant cell systems infected with recombinant virus expression vectors (e.g., cauliflower mosaic virus CaMV; tobacco mosaic virus, TMV) or transformed with recombinant plasmid expression vectors (e.g., Ti plasmid) containing the capsid protein coding sequences.

Two specific protocols for expressing and purifying core protein are described in detail in Example 2.

Nanoparticle Assembly

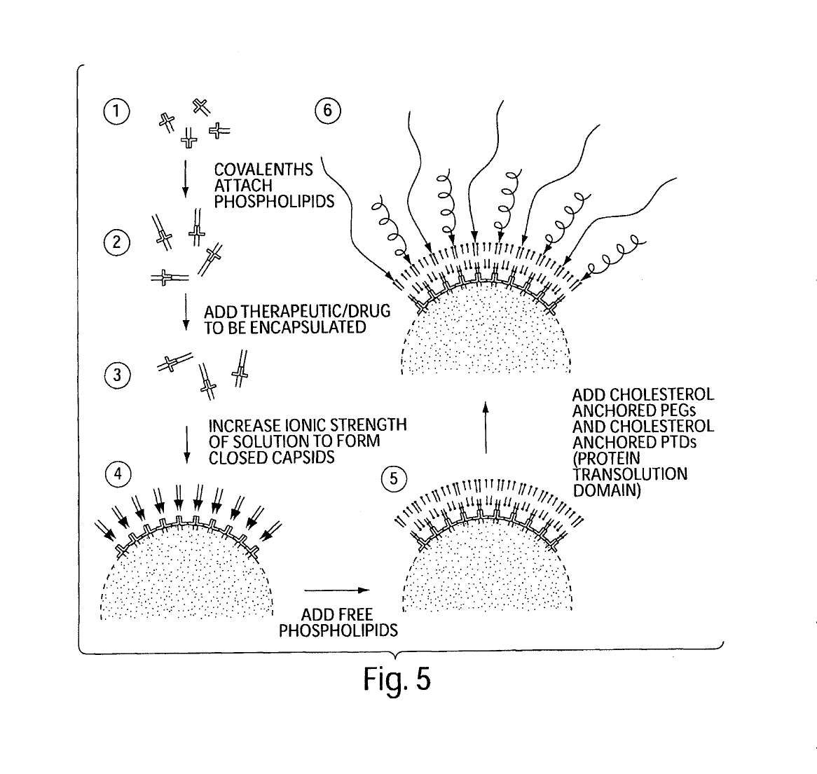

FIG. 5 is a flow diagram showing a general overview of one method of forming a self-assembling nanoparticle drug delivery system. The specific steps of FIG. 5 are described:

1. Mixing the appropriate engineered C-protein with the bioactive agent of choice;

2. Increasing the ionic strength of solution with the addition of NaCl to form cages, encapsulating the bioactive agent inside;

3. Adding engineered S-protein or engineered peptide to the cages;

4. Adding sonicated phospholipids solution to the mixture;

5. Adding cholesterol or lipid-tagged polyethylene glycol to the mixture;

6. Adding cholesterol or lipid-tagged protein transduction domains to the mixture; and

7. Purifying the system by centrifugation or size exclusion chromatography.

Thus, the bioactive agent is incorporated into the nanoparticle drug delivery system during the assembly of the cage. Core protein in a mildly buffered solution is mixed with an appropriate bioactive agent. As will be well known to those skilled in the art, any buffer system compatible with both C-protein and the bioactive agent can be used. Examples of suitable buffers include, but are not limited to, phosphate, citrate and Tris buffers as well as other buffers well known to those skilled in the art. In one example, protein drugs can be encapsulated in protein nanocages. Nanocages comprised of HBV C-protein can be packed with up to 1200 copies of a 10 kDa protein or an equivalent amount of at least one of a protein, peptide, nucleic acid or small molecule synthetic chemical entity. Therapeutic protein:C-protein complexes form in just a few seconds after mixing as dictated by the general physics of molecular diffusion and coulombic attraction.

To prevent the premature formation of the capsid, the capsid proteins are maintained in any suitable chemical denaturant or denaturing agent known in the art (e.g., urea, guanidine hydrochloride (GuHCl), sodium dodecyl sulfate (SDS)) in a concentration of about 1M to about 6M, about 1.5M to about 5M, about 1.75M to about 4.5M, or any integer disposed within said ranges. In some embodiments, the chemical denaturant or denaturing agent is urea. The urea can be present in a concentration of about 2M to about 6M, about 3M to about 5M, about 3.5M to about 4.5M, or any integer disposed within said ranges. In some embodiments, the denaturant is in a concentration of about 4M. To trigger the self-assembly reaction of the capsid, the ionic strength of the solution is raised to a final concentration of about 50 mM to about 600 mM. The final concentration can be about 100 mM to about 550 mM, about 150 mM to about 500 mM, about 200 to about 450 mM, about 250 mM to about 400 mM or about 300 mM to about 350 mM, or any integer disposed within said ranges. The final ionic concentration of the solution is directly related to the amount of chemical denaturant present in the solution. An increase in ionic concentration will decrease the chemical denaturant concentration to about 0.5M to about 4M, about 0.5M to about 3M, about 0.5M to about 2M, or any integer disposed within said ranges. In some embodiments where the chemical denaturant is urea, it is present in a concentration of about 1M to about 4M, about 1M to about 3M, about 1M to about 2M, or any integer disposed within said ranges. A higher concentration of chemical denaturant present in the original solution will necessitate a higher concentration of ionic strength to trigger self-assembly of the capsid.

In addition to salt and chemical denaturant concentrations, temperature can facilitate self-assembly of the capsid. A temperature of about 25° C. to about 105° C., about 40° C. to about 90° C. or about 55° C. to about 75° C. (or any specific temperature within the recited ranges) can trigger self-assembly of the capsid.

After incubating the mixture, the presence of fully formed capsids is verified using standard biochemical analyses known in the art. The cage is then mixed with any bifunctional linker or activated lipid known in the art that can facilitate the lipid coating of the cage. Alternatively, the linker can be re-engineered S-protein or a transmembrane engineered peptide as shown in FIG. 5. These additions can be covalently linked to a complementary cysteine on the surface of the cage at the spike of each building block.

Phospholipids can be incorporated into the C-protein matrix. The most stable association involves covalently combining a phospholipid to a functional group found on the side chains of specific amino acids within the C-protein. In the two protocols presented in Examples 3 and 4, heterobifunctional cross-linking molecules are utilized in order to provide a wide template for which many different functional groups found on different amino acids can be utilized, with the goal of optimizing distance constraints, solvent interactions, combinations of amino acid residue functional groups and phospholipids, and simplicity of synthesis. Examples 3 and 4 show the addition of sulfhydryl functional groups to the C-protein. Through these functional groups, phospholipid molecules can then be anchored which guide the coating process. Suitable ratios of protein:lipid for the coating process range from approximately 1:1 protein:lipid (w:w) to approximately 1:30 protein:lipid (w:w).

The use of heterobifunctional cross-linking molecules allows the possibility of engineering different functional groups at appropriate anchor points along the C-protein matrix while using the same phospholipid precursors, if necessary. For example, sulfhydryl functional groups are also involved in stabilizing the intermolecular interactions between core proteins that can stabilize the core cage. If utilizing the same functional group for anchoring phospholipids prevents the sulfhydryl functional groups from forming inter-molecular bonds and therefore negatively impacts the stability of the core protein shell, then other functional groups including, but not limited to, hydroxyl and amine groups, can be engineered into the protein at locations where phospholipid anchoring is specifically designed. This merely requires re-engineering the core proteins at a single location, and the use of an alternative, commercially-available heterobifunctional cross-linking molecule.

The coat layer of the nanoparticle can be a layer of neutral, cationic or anionic lipids alone or mixed with varying ratios of cholesterol. The layer can be a complex lipid coating material. The lipid layer can partially or completely coat the protein nanocage and can be single or multi-layered. The complex lipid coating material can be comprised of various amounts of phospholipids and cholesterol. Preferably, the complex lipid coating material is comprised of cholesterol, HSPC and POPG. A homogeneous mixture of various ratios of lipids (predominately phospholipids) and cholesterol can be made by adding dried components to a solution of chloroform:methanol (2:1 by volume). For example, and not intended as a limitation, 100 mg of phosphatidylcholine, 40 mg of cholesterol, and 10 mg of phosphatidyl glycerol are added to 5 mL of chloroform/methanol solution. This mixture is gently shaken to thoroughly mix all components. Next the mixture is dried down so as to remove all organic solvents. This dried mixture is then introduced to a few milliliters of aqueous solution (buffered H₂O) and mechanically dispersed by sonication. This solution is quickly added to a suspension of fully assembled nanocages containing captured drug payloads. The nanocages can already have been covalently modified with either coat enhancing peptides (engineered or S-protein) or with phospholipids. After a brief incubation with gentle mixing, coated cages are separated and purified using simple centrifugation and size exclusion chromatography.

Administration and Dosage

The nanoparticle drug delivery system can be administered by any conventional route and can be utilized to treat any disease or disorder for which a bioactive agent can be utilized. These include, but are not limited to the systemic routes, e.g. subcutaneous, intradermal, intramuscular or intravenous route, and mucosal routes, e.g. oral, nasal, pulmonary or anogenital route. When the treatment of solid tumors is involved, the intratumor route can also be used. When the treatment of genetic diseases is involved, the choice of the route of administration will essentially depend on the nature of the disease; for example, there can be administered via a pulmonary route in the case of cystic fibrosis (the nanoparticles being formulated in aerosol form) or via intravenous route in the case of hemophilia.

The nanoparticle drug delivery system can be used of regulate gene expression in a cell by administering or introducing a self-assembling nanoparticle drug delivery system containing bioactive molecule that can be iRNA, siRNA or shRNA (or a DNA encoding for iRNA, siRNA or shRNA), wherein the iRNA, siRNA or shRNA interferes with the mRNA of the gene to be regulated, thereby regulating expression of said gene. The cell can be in vitro, in vivo or ex vivo. The present invention also provides the use of the nanoparticle drug delivery system in the manufacture of a medicament for the regulation of gene expression or in the treatment of a disease, disorder or condition associated with the altered gene expression in a subject (e.g., human, mammal or an suitable animal), where expression of at least one gene of interest is regulated following administration or introduction of the self-assembling nanoparticle drug delivery system containing bioactive molecule that can be iRNA, siRNA or shRNA (or a DNA encoding for iRNA, siRNA or shRNA). The present invention further provides the use of a self-assembling nanoparticle drug delivery system comprising a capsid comprised of altered, mutated or engineered Hepatitis B Virus (HBV) core proteins, a bioactive agent captured in said capsid, and a complex lipid mixture coating said capsid, wherein the altered, mutated or engineered HBV core proteins are characterized by improved binding affinity of the bioactive agent to the carboxyl terminal portion of the HBV core proteins within the capsid for the treatment of a B cell malignancy or autoimmune disorder. The invention additionally comprises a nanoparticle drug delivery system as described in this application. Methods of regulating gene expression with iRNA, siRNA or shRNA are well known in the art. See, PCT Publication No. WO 06/066048, for example.

The nanoparticles of the nanoparticle drug delivery system can be administered in a biocompatible aqueous solution. This solution can be comprised of, but not limited to, saline or water and optionally contains pharmaceutical excipients including, but not limited to, buffers, stabilizing molecules, preservatives, sugars, amino acids, proteins, carbohydrates and vitamins. Suitable carriers are described in the most recent edition of Remington's Pharmaceutical Sciences, a standard reference text in the field, which is incorporated herein by reference.

For increasing the long-term storage stability, the nanoparticles of the nanoparticle drug delivery system can be frozen and lyophilized in the presence of one or more protective agents such as sucrose, mannitol, trehalose or the like. Upon rehydration of the lyophilized nanoparticles, the suspension retains essentially all drug previously encapsulated and retains the same particle size. Rehydration is accomplished by simply adding purified or sterile water or 0.9% sodium chloride injection or 5% dextrose solution followed by gentle swirling of the suspension. The potency of drug encapsulated in the nanoparticle is not lost after lyophilization and reconstitution.

The administration of nanoparticles can be carried out at a single dose or at a dose repeated once or several times after a certain time interval. The appropriate dosage varies according to various parameters, for example the therapeutically effective dosage is dictated by and directly dependent on the individual treated, the mode of administration, the unique characteristics of the bioactive agent and the particular therapeutic effect to be achieved, and the limitations inherent in the art of compounding such an active compound for the treatment of individuals. Appropriate doses can be established by persons skilled in the art of pharmaceutical dosing such as physicians. The nanoparticles can be included in a container, pack, or dispenser together with instructions for administration.

### EXAMPLES

Examples are provided below to further illustrate different features of the present invention. The examples also illustrate useful methodology for practicing the invention. These examples do not limit the claimed invention.

### Example 1

### 77C His-Tagged Core Protein

The 77C His-tagged Core Protein was cloned into the NdeI/XhoI restriction sites of vector pET21b (Novagen). This plasmid was transformed into E. coli BL21 (DE3) PlysS cells (Stratagene) for protein expression via normal methods. The nucleic acid and amino acid sequences are below.

77C His-tagged Core Protein has the following nucleic acid sequence:

(SEQID NO: 28)
ATG GAT ATC GAT CCG TAT AAA GAA TTT GGC GCC ACC GTG GAA CTG CTG AGC TTT
CTG CCG AGC GAT TTC TTT CCG AGC GTG CGT GAT CTG CTG GAT ACC GCG AGC GCG
CTG TAT CGC GAA GCG CTG GAA AGC CCG GAA CAT TGT AGC CCG CAC CAT ACC GCC
CTG CGT CAG GCG ATT CTG TGC TGG GGT GAA CTG ATG ACC CTG GCG ACC TGG GTT
GGC AAC AAC CTG TGT GAT CCG GCG AGC CGC GAT CTG GTT GTG AAC TAT GTG AAT
ACC AAC ATG GGC CTG AAA ATT CGT CAG CTG CTG TGG TTT CAT ATC AGC TGC CTG
ACC TTT GGC CGC GAA ACC GTG CTG GAA TAT CTG GTG AGC TTT GGC GTT TGG ATC
CGT ACC CCG CCG GCG TAT CGT CCG CCG AAT GCG CCG ATT CTG AGC ACC CTG CCG
GAA ACC ACC GTT GTG CGT CGC CGT GGT CGC AGC CCG CGC CGT CGT ACC CCG AGC
CCG CGT CGT CGT CGT AGC CAG AGC CCG CGT CGT CGC CGC AGC CAG AGC CGC GAA
AGC CAG CTC GAG CAC CAC CAC CAC CAC CAC

77C His-tagged Core Protein has the following amino acid sequence:

(SEQ ID NO. 29)
MDIDPYKEFGATVELLSFLPSDFFPSVRDLLDTASALYREALESPEHCSPHHTALRQAILCWGELMTLATWVGN
NLCDPASRDLVVNYVNTNMGLKIRQLLWFHISCLTFGRETVLEYLVSFGVWIRTPPAYRPPNAPILSTLPETTV
VRRRGRSPRRRTPSPRRRRSQSPRRRRSQSRESQLEHHHHHH

Poly-lysine Core Protein Mutants:

PCR: DNA fragments containing the genes for K5, K7, K9, K10, K11, K13, K20, KA9, KG9 and K4-5 core protein mutants were synthesized via PCR using the Cassette1 template and the primer sequences described in Table 1. Each PCR reaction was composed of 12.5 μl of 5×GC polymerase buffer (Finnzyme), 1.25 μl of a 10 mM dNTP mixture, 1.5 μl of 5 μM forward primer, 1.5 μl of 5 μM reverse primer, 0.6 μl of Stratagene mini-prepped template, 0.8 μl of 2 unit/μl Phusion Hot Start polymerase (Finnzyme), and 44.25 μl of water. The PCR reaction consisted of a one-time incubation at 98° C. for 1 minute, followed by incubation at 98° C. for 25 seconds, incubation at 70° C. for 30 seconds, and incubation at 72° C. for 1 minute and 10 seconds. These last three steps were repeated 24 times followed by a final incubation at 72° C. for 7 minutes.

The Cassette1 template consists of following nucleic acid sequence inserted into the NdeI/XhoI restriction site of vector pET22b:

(SEQ ID NO. 30)
ATGGATATCGATCCGTATAAAGAATTTGGCGCCACCGTGGAACTGCTGAGCTTTCTGCCGAGCGATTTCTTTCC
GAGCGTGCGTGATCTGCTGGATACCGCGAGCGCGCTGTATCGCGAAGCGCTGGAAAGCCCGGAACATTGTAGCC
CGCACCATACCGCCCTGCGTCAGGCGATTCTGTGCTGGGGTGAACTGATGACCCTGGCGACCTGGGTTGGCAAC
AACCTGTGCGATCCGGCGAGCCGCGATCTGGTTGTGAACTATGTGAATACCAACATGGGCCTGAAAATTCGTCT
GCTGCTGTGGTTTCATATCAGCTGCCTGACCTTTGGCCGCGAAACCGTGCTGGAATATCTGGTGAGCTTTGGCG
TTTGGATCCGTACCCCGCCGGCGTATCGTCCGCCGAATGCGCCGATTCTGAGCACCCTGCCGGAAACCACCGTT
GTCGACAAGCTTGCGGCCGCACTCGAGCACCACCACCACCACCACTGA

Ligation: The PCR products and a pET22b vector were both digested with restriction enzymes NdeI and XhoI at 37° C. for 2 hours. The digested products were run on an agarose gel, the bands excised, and purified via gel extraction (Stratagene). Ligation reactions were composed of 5 μl of digested and purified PCR product, 1 μl of digested and purified pET22b vector, 1 μl of T4 DNA ligase buffer (NEB), 1 μl of T4 DNA ligase (NEB), and 2 μl of water and were incubated at room temperature for 12 hours.

Transformation and DNA Sequencing: The ligation reactions were transformed into XL1 Blue E. coli cells (Stratagene) and the resulting colonies were grown in 1×LB broth and the plasmid purified via mini-prep (Stratagene). The purified plasmids were sequenced (see below) and transformed into E. coli BL21 (DE3) PlysS cells (Stratagene) for protein expression. This strategy can be used for proteins containing from 0 to 30 lysine residues. The nucleic acid and amino acid sequences for K5, K7, K9, K10, K11, K13 and K20 core protein mutants are described herein.

### Example 2

The various wild type and modified core proteins described herein can be expressed and purified according to Protocol 1 or Protocol 2 as follows:

### Protocol 1:

A pET-11a vector containing the full-length HBV C-protein gene, is transformed into E. coli DE3 cells and grown at 37° C. in LB media, fortified with 2-4% glucose, trace elements and 200 ug/mL carbenicillin. Protein expression is induced by the addition of 2 mM IPTG (isopropyl-beta-D-thiogalactopyranoside). Cells are harvested by pelleting after three hours of induction. SDS-PAGE is used to assess expression of C-protein.

Core protein is purified from E. coli by resuspending in a solution of 50 mM Tris-HCl, pH 7.4, 1 mM EDTA, 5 mM DTT, 1 mM AEBSF, 0.1 mg/mL DNasel and 0.1 mg/mL RNase. Cells are then lysed by passage through a French pressure cell. The suspension is centrifuged at 26000×G for one hour. The pellet is discarded and solid sucrose added to the supernatant to a final concentration of 0.15 M and centrifuged at 100000×G for one hour. The pellet is discarded and solid (NH₄)₂SO₄ is then added to a final concentration of 40% saturation, stirred for one hour and then centrifuged for one hour at 26000×G. The pellet is resuspended in a solution of 100 mM Tris-HCl at pH 7.5, 100 mM NaCl, 50 mM sucrose and 2 mM DTT (Buffer A) and loaded onto a Sepharose CL-4B (Pharmacia Biotech, Piscataway, N.J.) column (5 cm diameter×95 cm) equilibrated with Buffer A. and the column eluted at 2 mL/minute. Using this purification scheme, HBV viral capsids are separated from large aggregates and from soluble proteins of lower molecular weight. The fractions are pooled according to chromatographic profile and SDS-PAGE analysis and the solution concentrated by ultrafiltration using Diaflo YM 100 ultrafitration membrane (Amicon, Beverly, Mass.) to about 10 mg/mL. Concentrated C-protein is dialyzed against 50 mM Tris-HCl, pH 7.5 and 0.15 M sucrose. The solution is then adjusted to pH 9.5 with 10N NaOH and urea added to a final concentration of 3.5 M. The solution is then filtered using a Millex-HA 0.45 um pore size filter unit (Millipore, Bedford, Mass.) and applied to a column (6.0 cm diameter×60 cm) of Superdex 75 (Pharmacia Biotech, Piscataway, N.J.) equilibrated with 100 mM sodium bicarbonate, pH 9.5, containing 2 mM DTT. The column is eluted at 5 mL/minute. The fractions containing dimeric protein as assessed by SDS-PAGE are pooled. These procedures can be used for the expression and purification of all core protein mutants. Alternately, the expression of this protein can be done in yeast cells according to methods well known to persons skilled in the art.

### Protocol 2:

All protein constructs containing a C-terminal 6-histidine tag were purified as follows:

Starter Culture: The pET vector containing the gene for K9 protein is kept in BL21 (DE3) PlysS cells for expression. The starter culture can be inoculated from a colony on an 1× Luria Broth (1×LB) agar plate or from a 10% glycerol stock, stored at −80° C. Autoclave 1×LB in a 2 L flask. Let cool, then add 100 mg of ampicillin (Amp). Inoculate culture and allow to grow for up to 24 hours shaking at 200 rpm at 37° C.

Cell Growth and Isolation: Autoclave fifteen 2 L flasks with 0.8 L of 2× yeast-tryptone (2×YT) broth. Add 1 mL of 100 mg/mL ampicillin to each flask. Add 50 mL of starter culture to each flask. Incubate at 37° C., while shaking at 200 rpm until the optical density (OD) at 600 nm reaches 0.4-0.6. This process should take approximately 2 hours. When the OD reaches 0.4-0.6, induce with 1 mL of 1 M IPTG. Continue shaking for 4 more hours (OD will reach 2.0 or greater). Harvest the cells by centrifuging in 500 mL centrifuge bottles at 11300 g for 8 minutes. Transfer the bacterial pellets into two 50 mL conical tubes. Label each tube with date/construct/prep number and freeze at −20° C.

Cell disruption protocol: Thaw out two 50 ml tubes (approximately 20 mL each) of cell paste. The following steps apply to each of the 2 tubes. Into each tube, add 40 mL resuspension buffer (4 M urea, 50 mM NaHCO₃ (pH 9.5), 10 mM imidazole). Resuspend cells by continuous pipetting. Pour resuspended cells into a 400 mL beaker and adding more resuspension buffer until there is ˜100 mL total cell resuspension in the beaker. Place beaker containing resuspended cells in an ice bath. Using a Branson probe sonifier on pulse mode at approximately 40% duty cycling, and power setting of 5, sonicate for 5 minutes. The cell mixture should be sonicated in several intervals, allowing it to rest on ice in-between, if it appears that the sample can be heating to higher than room temperature. The cell lysate should be diluted by half to 200 mL total, and 100 μL of 100 mg/mL DNase should be added to the suspension. Let this suspension stir while on ice for 10 minutes. Repeat the sonication step for 5 more minutes while still on ice. Transfer the lysate to six 50 mL plastic centrifuge tubes, and centrifuge at 32000 g for 45 minutes. Decant off supernatant and save.

Nickel Column Purification Protocol: A 50 mL Ni²⁺-NTA agarose (Qiagen) column should be washed and equilibrated in the resuspension buffer. A full 12 L cell growth should be lysed for each run of the column. The centrifuged lysate from 12 L worth or cells should be combined and diluted to 500 mL with resuspension buffer. Load centrifuged cell lysate onto the column, and allow protein solution to sink to the top of the nickel matrix. Pass 50 mL of resuspension buffer through the column. Save the flow through in the event that the protein does not bind to column. An optional salt wash can be performed here by washing the column with 250 mL of NaCl wash buffer (4 M urea, 50 mM NaHCO₃ (pH 9.5), 20 mM imidazole, 250 mM NaCl). This salt wash reduces the A₂₆₀/A₂₈₀ ratio of the final purified protein by a value of 0.1 A.U. Wash column with 250 mL of wash buffer (4 M Urea, 50 mM NaHCO₃ (pH 9.5), 20 mM imidazole). Save the wash in the event that the protein does not bind to column. Pass 200 mL of elution buffer (4 M Urea, 50 mM NaHCO₃ (pH 9.5), 250 mM imidazole) through the column. Collect every 20 mL, which should yield 4 to 5 fractions that contain protein.

Measure Concentration and Dialysis: Measure the absorbance of the fractions to detect for presence and/or concentration of protein. Perform SDS polyacrylamide gel electrophoresis (SDS PAGE) analysis on protein to determine purity. Pool fractions containing K9 protein, and transfer to dialysis tubing. Dialyze into 4 L of storage buffer (4 M Urea, 20 mM NaHCO₃ (pH 9.5)) for at least 4 hours at 4° C. Repeat once. A 12 L cell growth yields approximately 500 mg of pure protein. Pure dialyzed protein can be stored at −80° C. for 6-8 months.

### Example 3

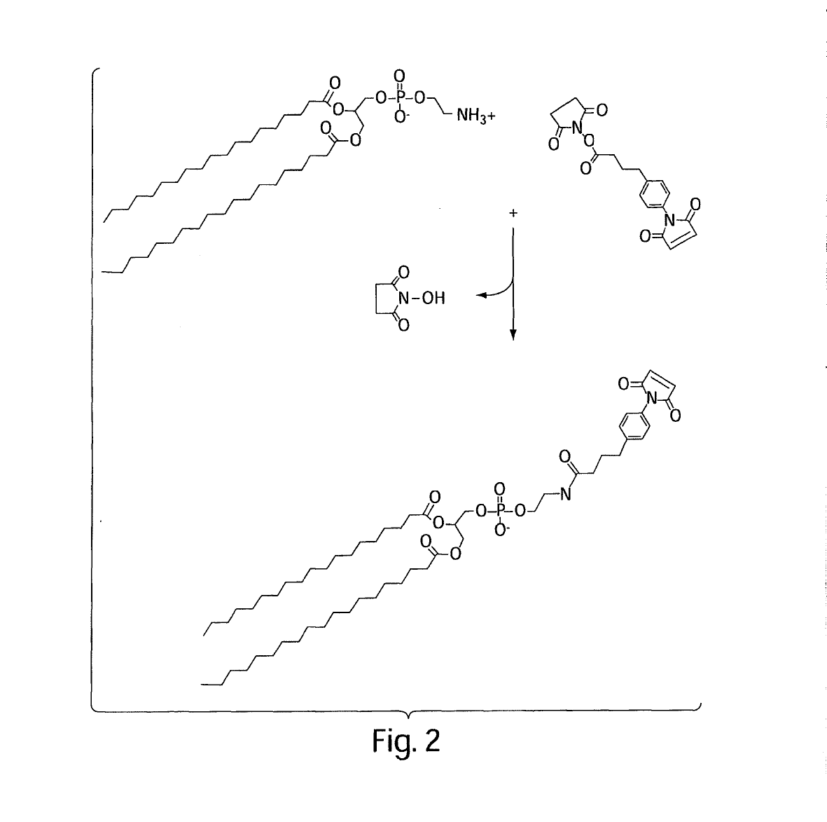

The following protocol describes conjugation of phospholipids via a SMPB (succinimidyl-4-(p-maleimidophenyl)butyrate) intermediate. This is shown schematically in FIG. 2.

1. Dissolve 100 micromoles of phosphatidyl ethanolamine (PE) in 5 mL of argon-purged, anhydrous methanol containing 100 micromoles of triethylamine (TEA). Maintain the solution under an argon or nitrogen atmosphere. The reaction can also be done in dry chloroform.

2. Add 50 mg of SMPB (Pierce) to the PE solution. Mix well to dissolve.

3. React for 2 hours at room temperature, while maintaining the solution under an argon or nitrogen atmosphere.

4. Remove the methanol from the reaction solution by rotary evaporation and redissolve the solids in chloroform (5 mL).

5. Extract the water-soluble reaction by-products from the chloroform with an equal volume of 1% NaCl. Extract twice.

6. Purify the MPB-PE derivative by chromatography on a column of silicic acid (Martin F J et al, Immunospecific targeting of liposomes to cells: A novel and efficient method for covalent attachment of Fab′ fragments via disulfide bonds. Biochemistry, 1981; 20:4229-38).

7. Remove the chloroform from the MBP-PE by rotary evaporation. Store the derivative at −20 C under a nitrogen atmosphere until use.

### Example 4

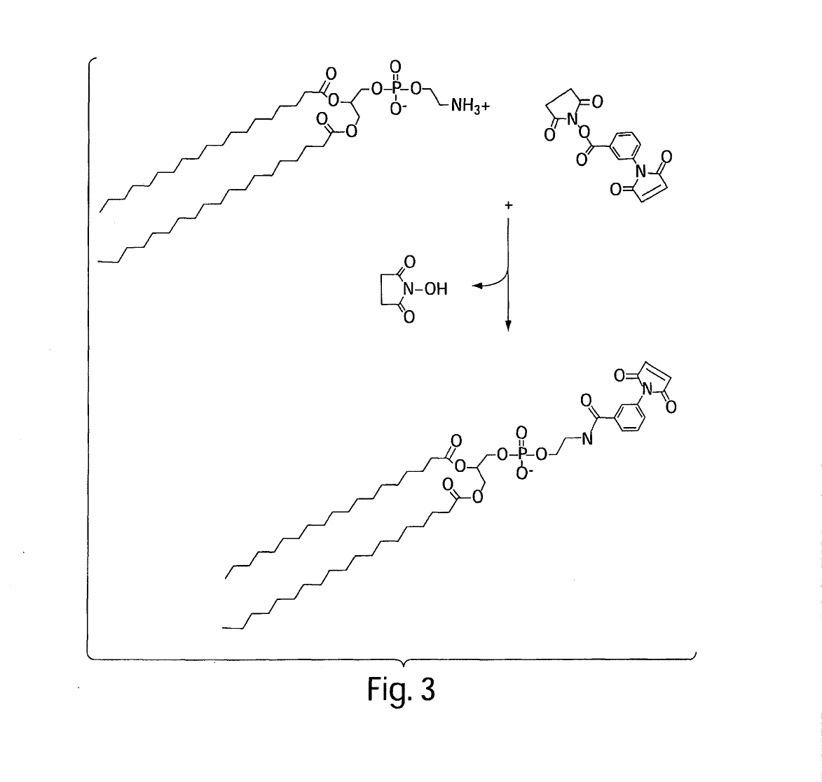

The following protocol describes conjugation of phospholipids via a MBS (m-maleimidobenzoyl-N-hydroxysuccinimide ester) intermediate. This is shown schematically in FIG. 3.

1. Dissolve 40 mg of PE in a mixture of 16 mL dry chloroform and 2 mL dry methanol containing 20 mg triethylamine, maintain under nitrogen.

2. Add 20 mg of MBS to the lipid solution and mix to dissolve.

3. React for 24 hours at room temperature under nitrogen.

4. Wash the organic phase three times with PBS, pH 7.3, to extract excess cross-linker and reaction by-products.

5. Remove the organic solvents by rotary evaporation under vacuum.

### Example 5

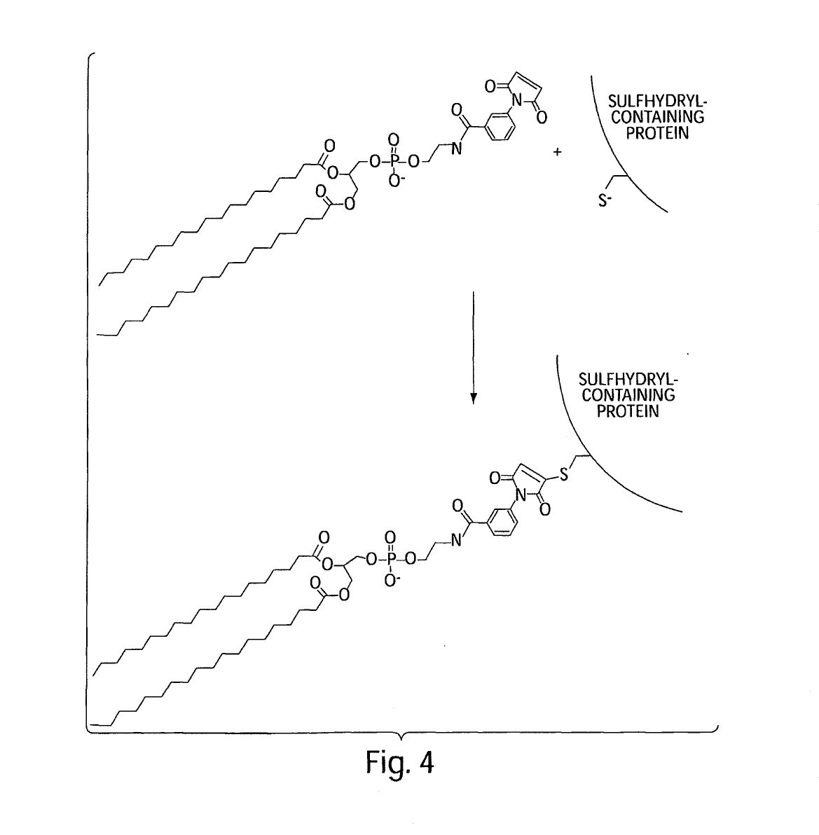

The following protocol describes conjugation of maleimide-containing intermediates (MCI) to sulfhydryl-containing proteins (SCP). This is shown schematically in FIG. 4.

1. Dissolve the SCP in TRIS*HCl buffer (pH=8.0, 100 millimolar) to obtain a concentration of 1 millimolar). Purge under a nitrogen or argon atmosphere for 20 minutes.

2. Dissolve the MCI in the same buffer as above, also purge under a nitrogen or argon atmosphere for 20 minutes, to obtain a 10-fold molar excess.

3. Combine the two solutions, and continue purging the solution under a nitrogen or argon atmosphere for an additional 20 minutes.

4. Allow the reaction to proceed for 6 hours, at room temperature.

### Example 6

The instant example describes a general method for forming the nanoparticle delivery system.

Prepare protein, add encapsulate, and form delivery system:

Add BME (betamercaptoethanol) to protein solution to get final concentration to 5 μM. Filter with 0.22 μm PES filter (Nalgene).
A. If encapsulating with DOX (Doxorubicin HCl), add predissolved encapsulate in ddH₂O to protein solution to obtain a final DOX solution of 0.5 mg/mL). Keep this solution in H₂O bath set to 25° C. for 12 hours.
B. If encapsulating siRNA, add the siRNA-containing solution to protein solution at a 3150× molar excess (nucleic acid:protein monomer). Add a solution of 0.5 M NaCl to solution to obtain final NaCl concentration of about 50 mM to about 150 mM (additional ranges include about 50 mM to about 100 mM, about 50 mM to about 75 mM, about 80 mM to about 150 mM, or any integer disposed within these ranges). Keep this solution in H₂O bath set to 25° C. for 12 hours.

First, FPLC (fast performance liquid chromatography) purification: Purify cage material via FPLC (Amersham Pharmacia). The large FPLC column (Pharmacia XK-26 26 mm×1000 mm) can be run at 1.5 mL/min running 0.5×PBS pH 9.4 buffer as the mobile phase, Sepharose CL-4B (Amersham Pharmacia) matrix as the stationary phase. Collect and combine delivery system fractions and run a gel (SDS-page; Biorad) to determine the delivery system concentration versus protein standards (usually made with just CpB1 protein in dialysis buffer). Cross-reference the protein concentration with an absorbance measurement at 280 nm. Concentrate protein solution to 1.0 mg/mL via the Amicon filtration system.

Make lipid coating material: Premix cholesterol (Avanti Lipids, Alabaster, Ala., USA) and HSPC (L-α-Phosphatidylcholine, Hydrogenated (Soy), Avanti Lipids, Alabaster, Ala., USA) and DiI (1,1′-Dioctadecyl-3,3,3′,3′-tetramethylindocarbocyanine perchlorate; Sigma Aldrich, St. Louis, Mo., USA) in a 31.9:15.6:1 molar ratio, respectively, as dry powders in a glass beaker. Predissolve and homogenize with 2.0 mL of chloroform. Once homogenized, evaporate off the chloroform (20 to 30 minutes on a hot plate set to 50° C.). Once dry, add 0.5×PBS to make the lipid coating material at a concentration of 0.2 mg/mL. Probe sonicate this solution (240 seconds, power level=7, cycle=50%). Mix this aqueous lipid coating material solution at 70° C. for an additional 30 minutes.

Functionalize protein with maleimide-terminated lipid: Treat the raw cage solution with TCEP (tris-carboxyethylphosphine) as a dry powder in a 4-fold molar excess compared to the protein concentration (1 exposed sulfhydryl per CpB1 protein; 240 exposed sulfhydryls per cage). Add PE-MAL (1,2-Dipalmitoyl-sn-Glycero-3-Phosphoethanolamine-N-[4-(p-maleimidophenyl)-butyramide] (Sodium Salt)) in 3× molar excess predissolved in 500 μl DMF (dimethylformadmide) dropwise to the raw cage solution. Allow the PE-MAL to react with the raw cage for 60 seconds.

Coat the functionalized cage, and purify via FPLC: After which, add the lipid coating material solution to the functionalized cage solution at a mass ratio of 1:3. Allow to mix and homogenize for 60 minutes by stirring and heating on hot plate at 60° C. Filter once with 0.45 μm Whatman PES filter (almost all of the material should pass easily through the filter). Repeat with a 0.22 μm Nalgene PES filter (again, almost all of the material should pass through the filter relatively easily). Purify this material via FPLC with 0.5×PBS buffer, pH 9.4. Again, the coated cage elutes from 220 to 280 mL. Collect the fractions verify the delivery system size via dynamic light scattering (Dynapro Titan, Wyatt Instruments, Goleta, Calif.) and obtain concentration via SDS-page gels.

As a non-limiting example, a specific protocol for forming a lipid coated nanocage encapsulating a bioactive agent is as follows:

Protein Expression and Purification:

Express viral core proteins in E. coli using common microbiology methods as described herein. Disrupt the bacteria by sonication in a basic denaturing solution comprising 4M urea, 50 mM NaHCO₃, 10 mM imidazole at pH 9.5 and 100 μL of 100 mg/mL DNase. Subject the solution to sonication five more times after DNase treatment. Centrifuging the sonicated solution to pellet insoluble matter, removing the soluble matter (supernatant) and loading the soluble matter onto a nickel-agarose column. Wash the column with two column volumes of 4M urea, 50 mM NaHCO₃, 10 mM imidazole at pH 9.5. Additionally wash the column with ten column volumes of 4M urea, 50 mM NaHCO₃ 20 mM imidazole at pH 9.5. Elute the protein with more than four column volumes of 4M urea, 50 mM NaHCO₃, 250 mM imidazole at pH 9.5.

Nanocage Formation:

Add beta mercaptoethanol and the bioactive agent to be captured to the protein eluted from the column. Increase the ionic strength of the solution and decease the urea concentration to 2M by adding salt (NaCl) to a final salt concentration of 0.5 M. The process of nanocage formation and capture of the bioactive agent must proceed under conditions that are free or substantially-free of nucleases (e.g., DNAse, RNAse) and proteinases to ensure that the bioactive agent is not damaged or degraded. Substantially-free as used herein means that DNA, RNA or protein is not damaged or degraded by the presence of an nuclease or proteinase present prior to encapsulation in the capsid such that it is no longer therapeutically effective.

Purification:

Purify the nanocage using fast performance liquid chromatography (FPLC using a solid phase of either CL2B or CL4B and using a purification mobile phase of 0.5 M PBS buffer at pH 9.4 or pH 7.2. Concentrate the purified nanocage using amiconfiltration to a final concentration of 1 mg/mL.

Lipid Coating:

Treat the purified nanocage with TCEP or PE-Mal. The PE-Mal can be coated with a lipid composite material. The lipid composite material can be composed of: (a) 20% Cholesterol and 80% HSPC; (b) 50% Cholesterol and 50% HSPC; (c) 20% Cholesterol and 20% HSPC and 60% POPG; (d) 50% Cholesterol and 50% POPG; (e) 20% Cholesterol and 80% POPG; or (f) 10% Cholesterol and 15% HSPC and 65% POPG. Preferably, the lipid composite material is 20% Cholesterol and 20% HSPC and 60% POPG. For fluorescent verification of lipid coat add 3% DiI by mole ratio to the lipid composite material. Homogenize the lipid composite material in chloroform and then remove the chloroform. Resuspend the lipid composite material is resuspended in 0.5 M PBS buffer at pH 9.4 or at pH 7.2. Sonicate the lipid composite material. Add the lipid composite material to the purified nanocage treated with PE-Mal. Sonicate the mixture and then heat the mixture at 50° C. for 1 hour. Purify the mixture using FPLC using a solid phase of either CL2B or CL4B and using a purification mobile phase of 0.5 M PBS buffer at pH 9.4 or pH 7.2. Concentrate the purified nanocage using amiconfiltration to a final concentration of 1 mg/mL.

Targeting:

Treat the purified coated nanocage with a modified antibody. The modified antibody can be treated with Traut's reagent and further treated with PE-Mal. Purify the modified antibody using a g-50 solid phase and a purification mobile phase of 0.5 M PBS buffer pH 7.2. The antibody concentration is in excess by 20 mole equivalent to purified coated nanocage. Purify the coated nanocage comprising a modified antibody by FPLC using a solid phase of either CL2B or CL4B and using a purification mobile phase of 0.5 M PBS buffer at pH 9.4 or pH 7.2. Concentrate the purified nanocage using amiconfiltration to a final concentration of 1 mg/mL.

Specific nanoparticle assembly methods specific for various bioactive molecules are further described.

General Lipid Nanocage Assembly: Lipid nanocages assembled with K9 protein construct. Protein that was thawed is diluted with water to 2 M urea final concentration. To this solution is added 4 mole equivalents of betamercaptoethanol per protein molecule. This is then placed at 25° C. for 12 hours and the material is then treated with 4 mole equivalents per protein of 1,2-Dipalmitoyl-sn-Glycero-3-Phosphoethanolamine-N-[4-(p-maleimidophenyl)butyramide] (Sodium Salt) (PE-Mal) and then coated with 60:20:20 POPG:HSPC:CHOL [POPG (1-Palmitoyl-2-Oleoyl-sn-Glycero-3-[Phospho-rac-(1-glycerol)] (Sodium Salt), Avanti Lipids, Alabaster, Ala., USA), cholesterol (Avanti Lipids, Alabaster, Ala., USA) and HSPC (L-α-Phosphatidylcholine, Hydrogenated (Soy), Avanti Lipids, Alabaster, Ala., USA)] lipid coating at a mass value of 30% of the total protein. The coating material is prepared by sonicating the lipid coating material in 0.5×PBS pH 9.4 until it reaches 55° C. and then added to the protein solution. The subsequent mixture is then purified by FPLC.

Lipid Nanocage assembly with ssRNA: Lipid nanocages assembled with K9 protein construct. Protein that was thawed is treated with 0.1 mole equivalents to protein of ssRNA which is the antisense strand of siRNA. This is allowed to bind for 30 minutes and the solution is then diluted with water to 2 M urea final concentration. To this solution is added 4 mole equivalents of betamercaptoethanol per protein molecule. This is then placed at 25° C. for 12 hours and the material is then mixed with 4 mole equivalents per protein of PE-Mal and then coated with 60:20:20 POPG:HSPC:CHOL lipid coating at a mass value of 30% of the total protein. The coating material is prepared by sonicating the lipid coating material in 0.5×PBS pH 9.4 until it reaches 55° C. and then added to the protein solution. The subsequent mixture is then purified by FPLC.

Lipid Nanocage assembly with dsRNA: Lipid nanocages assembled with K9 protein construct. Protein that was thawed is treated with 0.1 mole equivalents to protein of dsRNA which can be 21 nt blunt end, 19 nt+2 nt overhang, or 27 nt blunt end. This is allowed to bind for 30 minutes and the solution is then diluted with water to 2 M urea final concentration. To this solution is added 4 mole equivalents of betamercaptoethanol per protein molecule. This is then placed at 25° C. for 12 hours and the material is then mixed with 4 mole equivalents per protein of PE-Mal and then coated with 60:20:20 POPG:HSPC:CHOL lipid coating at a mass value of 30% of the total protein. The coating material is prepared by sonicating the lipid coating material in 0.5×PBS pH 9.4 until it reaches 55° C. and then added to the protein solution. The subsequent mixture is then purified by FPLC. It was determined for the current method of loading siRNA that the ideal siRNA loading occurs at 24 siRNA strands per lipid coated nanocage which leads to, after purification, 10 siRNAs captured per lipid coated nanocage. At higher loading it is determined that lipid coated nanocage formation is can be limited.

Lipid Nanocage assembly with DNA Ladder: Lipid nanocages assembled with K9 protein construct. Protein that was thawed is treated with 0.1 mole equivalents to protein of DNA ladder (1 kb ladder) (N3232S). This is allowed to bind for 30 minutes and the solution is then diluted with water to 2 M urea final concentration. To this solution is added 4 mole equivalents of betamercaptoethanol per protein molecule. This is then placed at 25° C. for 12 hours and the material is then mixed with 4 mole equivalents per protein of PE-Mal and then coated with 60:20:20 POPG:HSPC:CHOL lipid coating at a mass value of 30% of the total protein. The coating material is prepared by sonicating the lipid coating material in 0.5×PBS pH 9.4 until it reaches 55° C. and then added to the protein solution. The subsequent mixture is then purified by FPLC.

Lipid Nanocages with PEG Lipid conjugates in the lipid coat: Lipid nanocages made from K9 protein and templated with PE-Mal, as mentioned above, were used to manufacture lipid nanocages with PEG lipids in the lipid coat. The lipid coat was composed in a ratio of 68:18:18:6, POPG:HSPC:CHOL:PEG lipid by mole ratios. The PEG lipids that are used in the lipid coating material are either 1,2-Distearoyl-sn-Glycero-3-Phosphoethanolamine-N-[Methoxy(Polyethylene glycol)-2000] (Ammonium Salt) or 1,2-Distearoyl-sn-Glycero-3-Phosphoethanolamine-N-[Methoxy(Polyethylene glycol)-350] (Ammonium Salt). The lipid coating material is sonicated to 55° C. and is added to the K9 lipid nanocages treated with PE-Mal and mixed and purified by FPLC.

Lipid Nanocage functionalization 4-Maleimidobutyric Acid (GMBA): Lipid nanocages were assembled with K9 protein construct. Protein was thawed and is treated with 10 mole equivalents per protein of 4-Maleimidobutyric Acid (GMBA). This is allowed to react for 30 minutes and is purified by FPLC. The purified lipid nanocages tested with Ellmans reagent to determine if any uncapped Cysteines are present on the surface.

### Example 7

### Methods of Providing Lipid Nanocage Targeting are Described

Antibody Modification for Delivery System Coupling: Antibodies at a concentration of 4 mg/mL in 1×PBS buffer pH 7.4 were treated with 20 mole equivalents of Traut's reagent, 2-iminothiolane HCl, for 1 hour. The antibodies were purified via column chromatography (8×200 mm) G-50 (Amersham Pharmacia) in 0.25×PBS buffer pH 7.4.

Delivery System Modification with antibodies: The delivery system was treated with 200 mole equivalents of PE-maleimide lipid (1,2-Dipalmitoyl-sn-Glycero-3-Phosphoethanolamine-N-[4-(p-maleimidophenyl)-butyramide] (Sodium Salt)) (dissolved in DMF) per mole equivalent of delivery system. Upon standing for 30 minutes the delivery system, 1 mole equivalent, was treated with 30 mole equivalents of antibodies modified with Traut's reagent (the above step). This was allowed to react overnight. Excess antibodies were purified from the antibody targeted system via a packed column (16×200 mm) packed with Sepharose CL-4B matrix with the isocratic mobile phase (0.25×PBS pH 7.4). This gives a typical yield of about 60% and has about 20-30 antibodies per delivery system as determined by SDS-PAGE gels.

### Example 8

### Transmission Electron Microscopy and Dynamic Light Scattering were Utilized to Assess and Validate Nanocage Formation

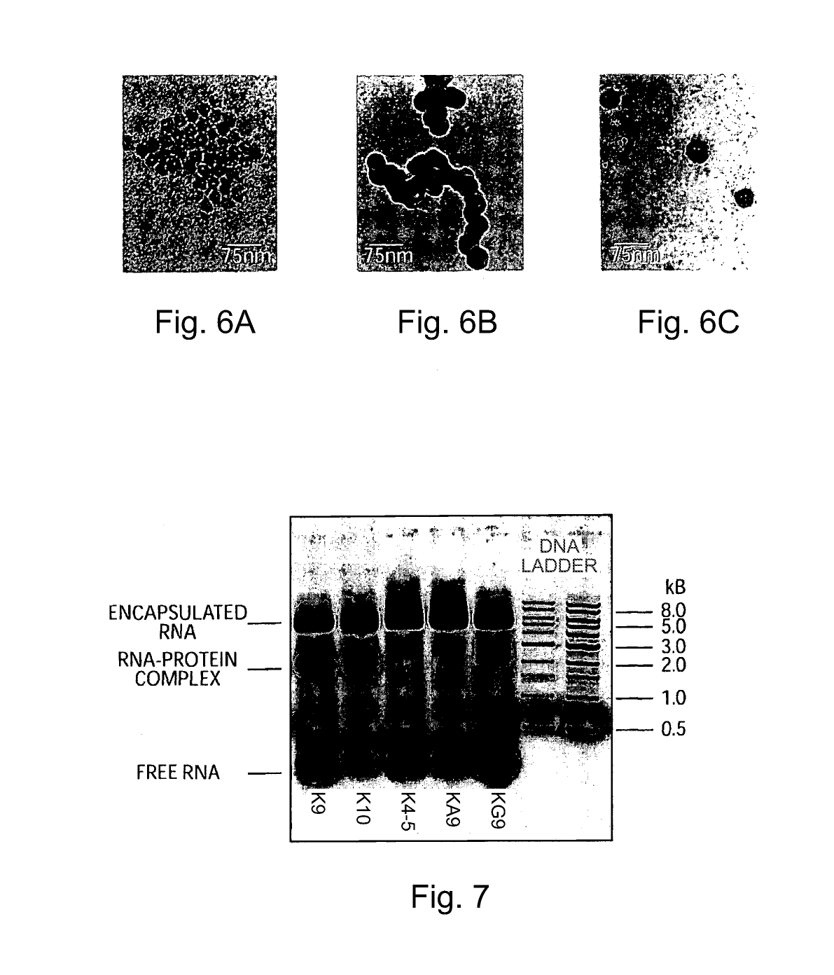

Transmission Electron microscopy (TEM) is a useful tool to examine the morphological characteristics of small (sub-micrometer) particles, including nanocages. As shown in FIG. 6, the structural details and extensive surface topology of nanocage particles are best revealed by the use of negative staining procedures. The negative staining process involves surrounding nanocages with electron-dense chemicals thus revealing the structure, size, and surface topology of individual particles as the contrast between the stain (dark) and the specimen (light). One “drop” of nanocage (100 ug/ml) in PBS was placed on multiple formvar coated copper mesh TEM grids (purchased from Electron Microscopy Sciences) followed by one drop of 1% PTA solution (phosphotungstic acid in water, pH adjusted to 7.0 with 1N NaOH). After 2 minutes, excess liquid was blotted with filter paper. TEM grids were then allowed to air dry for approximately 10 minutes. Grids were then examined using standard transmission electron microscopy (TEM). Photographs were taken at multiple magnifications (5000×-1,000,000×) using an attached digital camera. Multiple nanocage constructs were used for these experiments, including nanocages with and without attached anti-CD22 antibodies as well as naked nanocage particles lacking a lipid coat were also documented.

Dynamic Light Scattering (DLS) is a useful tool to examine the size characteristics of small (sub-micrometer) particles in solution. Solutions of purified nanoparticle drug delivery system was analyzed to validate that the predicted material was achieved at the end of the manufacturing process. Results indicate that select fractions from a size exclusion column used to purify final nanoparticle drug delivery systems were in fact very monodispersed.

ELISA is a useful tool in determining the ability of the nanocages to bind various bioactive agents. Protein constructs (3 mg/ml) were mixed with 200 uM RNA at a ratio of 6.25 protein dimers per RNA duplex and allowed to bind for 15 minutes. This mixture was then diluted 1:1 with 30 mM Sodium Hepes pH 7.5, 60 mM NaCl in order to encapsulate RNA. Encapsulation was allowed to proceed overnight at room temperature. The samples, along with a “1 kB” DNA ladder (New England Biolabs), were loaded on a 1.0% agarose gel, containing ethidium bromide, run for 40 minutes at 100 Volts, and visualized on a Molecular Dynamics Typhoon imager. The results in FIG. 7 show that RNA is readily encapsulated in the K9, K10, KA9, KG9 and K4-5 constructs to varying degrees.

### Example 9

The following assays describe the effectiveness and efficiency of various lipid nanocage systems.

Fluorescent Cage Binding Protocol (Anti-CD22 Targeted vs. Non-targeted Delivery Systems):

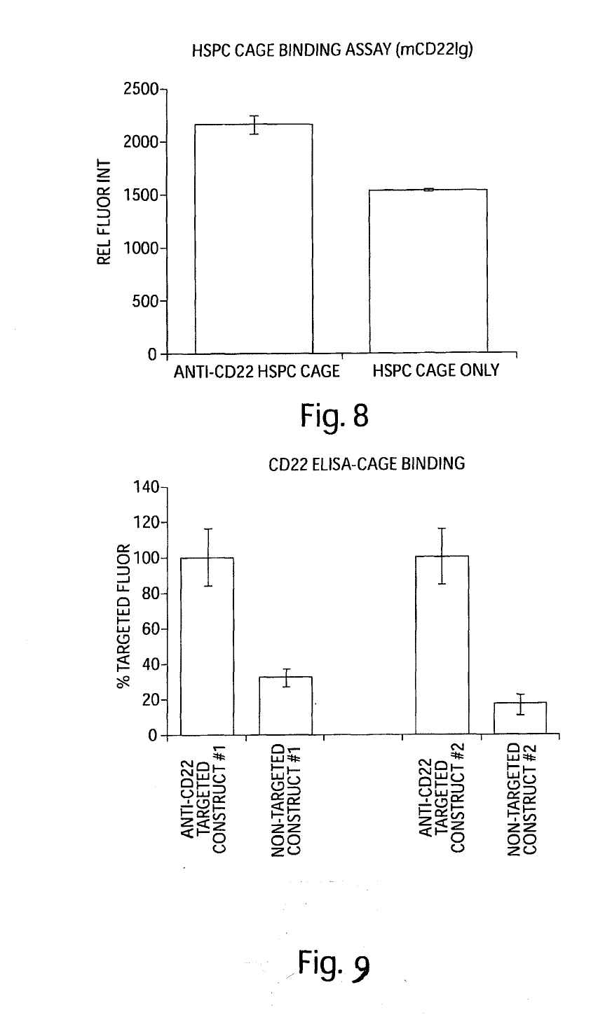

96-well ELISA plates were coated with either 50 μL of mCD22Ig protein or 2% BSA (w/v) in 0.1M borate buffered saline at a concentration of 50 ug/ml overnight. Plates were then washed 3 times in Tris buffered saline (TBS). All wells were then blocked with 2% BSA in TBS for 1 hour, followed by 3 TBS rinses. Anti-CD22 targeted cage constructs and non-targeted cage constructs (no antibody) containing 4% DiI embedded within the lipid coat were incubated in triplicate, at multiple concentrations, in buffer containing 2% BSA and 0.1% tween in TBS for 4 hours. Wells were then rinsed 4 times in TBS and plates were read using a Typhoon Molecular Imager (Molecular Dynamics). Background wells contained mCD22Ig (from original plating) and TBS. Fluorescent reads were conducted in TBS, averaged, standard deviations were calculated, and standard error of the means (error bars) calculated for each condition. The results shown in FIG. 8 show that fluorescently-labeled, antibody-targeted, lipid-coated cages bind to mCD22Ig significantly more than fluorescently-labeled, lipid coated non-targeted cages. Anti-CD22 HSPC cages bound 1.6 times better than HSPC cages only, indicating that delivery systems were targeted with antibodies.

Cage Binding ELISA (Anti-CD22 Targeted vs. Non-targeted Delivery Systems):

96-well ELISA plates were coated with either 50 μL of mCD22Ig protein or 2% BSA (w/v) in 0.1 M borate buffered saline at a concentration of 50 μg/mL overnight. Plates were then washed 3 times in Tris buffered saline (TBS). All wells were then blocked with 2% BSA in TBS for 1 hour, followed by 3 TBS rinses. Anti-CD22 targeted cage constructs and non-targeted cage constructs were incubated in triplicate, at multiple concentrations, in buffer containing 2% BSA and 0.1% tween in TBS for 4 hours. Wells were then rinsed 3 times in TBS followed by incubation in antibodies generated in- and against 1) rabbit-anti HBV core protein (AbCam), 2) mouse anti-HBV core protein (GenTex), or 3) no antibody in 2% BSA and 0.1% tween in TBS for 1 hour. Wells were then rinsed 3 times in TBS followed by 1 hour incubation in 1) goat anti-rabbit conjugated to alkaline phosphatase, 2) goat anti-mouse Fc region conjugated to alkaline phosphatase, or 3) no antibodies in 2% BSA and 0.1% tween in TBS. All wells were rinsed 3 times in TBS, one time in PBS, and incubated in DDAO-phosphate (1:100,000) in PBS. Primary antibodies (rabbit-anti HBV core protein (AbCam) or mouse anti-HBV core protein (GenTex)) were omitted in background control wells. Fluorescent reads were conducted using Cy5 excitation/emission settings on a Typhoon Molecular Imager, averaged, standard deviations were calculated, and standard error of the means (error bars) calculated for each condition (2 experiments included representing 2 cage preparations). Anti-core protein antibodies were used to detect the presence of nanocages. Non-targeted nanocage binding data are normalized to the % of anti-CD22 targeted nanocage binding. The results shown in FIG. 9 show that the mCD22Ig binding studies anti-CD22 HSPC cages bound 3.3 times better than non targeted cages only, indicating that delivery systems targeted with antibodies are more specific for a specific receptor. Similar results were obtained with an ELISA assay looking for core protein. In the core protein assay it was found that targeted delivery systems bound 5.6 times better than non targeted system.

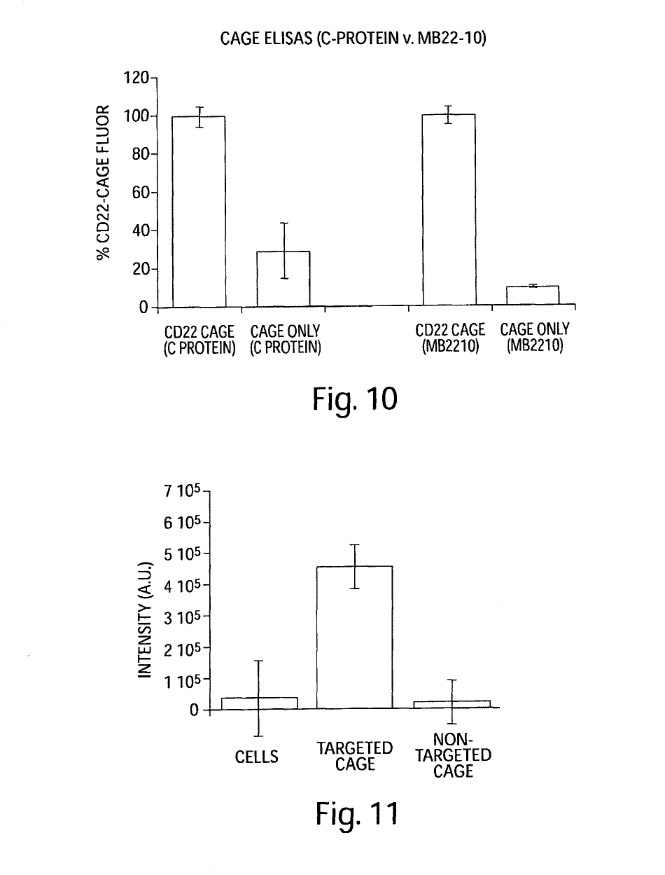

Separate ELISA′a were also conducted to measure the amount of mouse-anti CD22 antibody present on targeted cages versus non-targeted cages in each well (see above) using the same protocol but omitting the primary antibody step (rabbit-anti HBV core protein (AbCam) or mouse anti-HBV core protein (GenTex)). For these experiments, only goat anti-mouse Fc region specific antibodies were used to detect the presence of cages. DDAO-phosphate was used as the fluorescent substrate (see above) and all analyses were conducted in the same manner. Anti-core protein antibodies (blue columns) and goat-anti-mouse antibodies (red columns) were used to detect the presence of nanocages or anti-CD22 antibody on the surface of nanocages (respectively). Non-targeted nanocage binding data are normalized to the % of anti-CD22 targeted nanocage binding. The results in FIG. 10 show that the core protein assay it was found that delivery system bound 3.5 times better than non targeted system, indicating bind of antibodies to the delivery system surface. In the mCD22Ig binding studies anti-CD22 HSPC cages bound 9 times better than non targeted cages only, again indicating that delivery systems were targeted with antibodies are more specific for a specific receptor.

Cell Growth

B Cell (BCL1 and Ramos) and T cell lines (Jurkat and HH) were purchased from ATCC and grown at 37° C. (5% CO₂) in RPMI medium with 10% fetal bovine serum and supplements (as recommended by ATCC) including standard antibiotics. Cells consistently exhibited “normal” growth characteristics. All cell experiments were conducted while cells were exhibiting log-phase growth characteristics.

Fluorescent Cell Assays

Anti-CD22 Targeted vs. Non-targeted Fluorescent Cage Binding to Cells

9 mL Ramos cells (from cultures at a density of 1,000,000 cells/mL) were drawn from T75 culture flasks into 3 sterile 15 mL conical tubes (3 mL each), spun down, re-suspended in 3 mL complete RPMI medium (each). Cells were incubated with fluorescent anti-CD22 targeted cages, non-targeted cages (both with 3% Dil embedded in the lipid coat), or an equal volume of “media only” at 37° C. a concentration of 400,000 cages/cell in 3 mL (equal to ˜60 nM) for 2 hours. Cells were then spun down, rinsed 2 times in 5 mL complete media, rinsed 3 times in 5 mL sterile PBS, spun down and resuspended in 150 μL of PBS. 150 μL of 2% paraformaldehyde was then slowly added to the cells, cells were allowed to fix for 10 minutes, and 100 μL of cell suspension was added to each of 3 wells of a 96-well plate. Plates were then spun down using a clinical centrifuge and fluorescence was ready on Typhoon Molecular Imager using Cy3 excitation/emission settings. Fluorescent levels were averaged, standard deviations were calculated, and standard error of the means (error bars) calculated for each condition. Background fluorescence of “cells alone” is included for comparison. The results in FIG. 11 show that the targeted delivery systems get taken up by cells 3 times better than non targeted cages. Indicating that targeting with antibodies for CD22 improves cellular up take of the delivery system by B-cells.

Anti-CD22 Targeted Vs. Non-Targeted Fluorescent Cage Internalization

Adherent BCL1 cells were plated onto glass coverslips (Fisher Scientific) in sterile 24-well tissue culture plates 12 hours prior to experimentation. Cells were allowed to grow to semi-confluency (cell density estimated at 200,000 cells/well) in complete RPMI media (see Cell Growth above). Prior to experiments, cells were rinsed with once with media and 500 μl, of media was added to each well. Following experimental incubations (see below) adherent cells were rinsed, once in media and 3 times in PBS. Cells were then resuspended in 150 μL PBS and 150 μL of 2% paraformaldehyde was added to tubes to slowly fix cells.

A total of 200,000 suspension cells (Ramos, Jurkat, and HH Cells) were added to sterile 24-well tissue culture plates and media and volumes were adjusted (upwards) to 500 μL with complete media. Following experimental incubations (see below) suspension cells were sequentially pelleted and rinsed once in media and 3 times in PBS. Cells were then resuspended in 150 μL PBS and 150 μL of 2% paraformaldehyde was added to tubes to slowly fix cells.

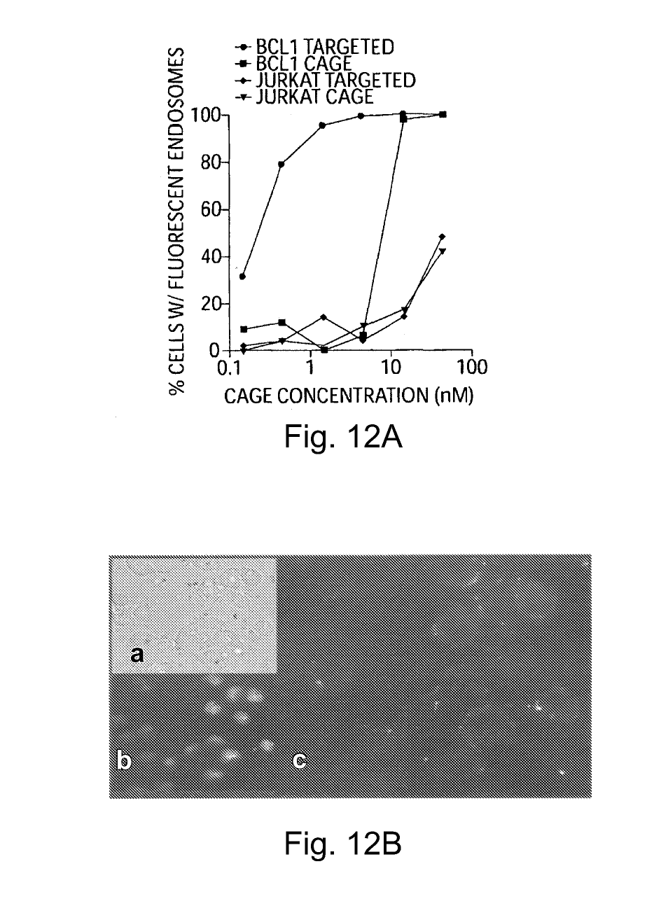

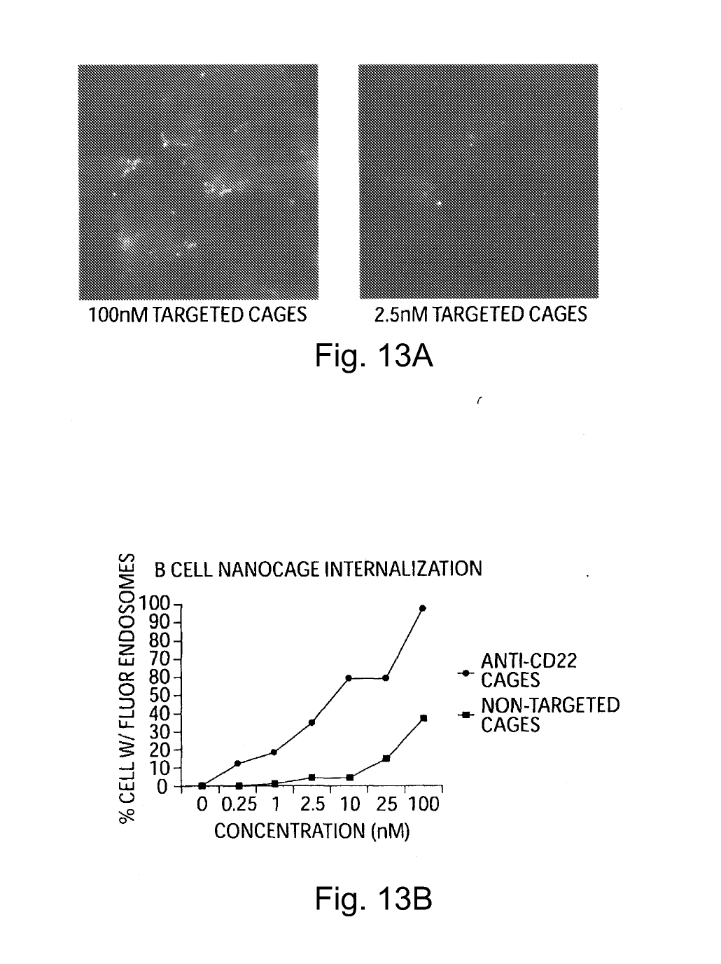

For experimental incubations, cells (adherent and suspension) were incubated with fluorescent anti-CD22 targeted cages, non-targeted cages (both with 3% DiI embedded in the lipid coat), or an equal volume of “media only” at 37° C. at multiple cage concentrations [300,000 cages/cell (˜30 nM), 100,000 cages/cell (˜10 nM), 30,000 cages/cell (˜3 nM), 10,000 cages/cell (˜1 nM), 3000 cages/cell (˜300 μM), and 1000 cages/cell (˜100 μM)] in 500 μL media for 2 hours. Following rinses and fixation (see above) cells were coverslipped wet in 5% n-propyl gallate in glycerol (w/v) and sealed under cover-slips using nail polish. Internalized fluorescent delivery systems were quantified using standard fluorescence microscopy. Two-hundred cells were counted per coverslip and the percentage of cells with internalized cages was quantified. The results in FIG. 12A show that non-targeted nanocages (nanocage) bind to both cell types with similar affinity at low concentrations, but to better to B Cells at higher concentrations. The results in FIG. 12B show that targeted delivery systems are preferentially internalized compared to non targeted delivery systems. Further, the targeted delivery system is specific for B-cells only when compared to similar dosage concentration used in T-cell experiments. Targeting of the delivery system significantly improves targeted cell uptake when compared to non-specific cells. The results in FIG. 13A show the internalization of anti-CD22 targeted nanocages and non-targeted nanocages in BCL1 cells at 100 nM and 2.5 nM dosages and the results in FIG. 13B show that targeted delivery systems are preferentially internalized compared to non targeted delivery systems.

Competition Assay Using Anti-CD22 Targeted Cages in the Presence of “Free-Anti-CD22”

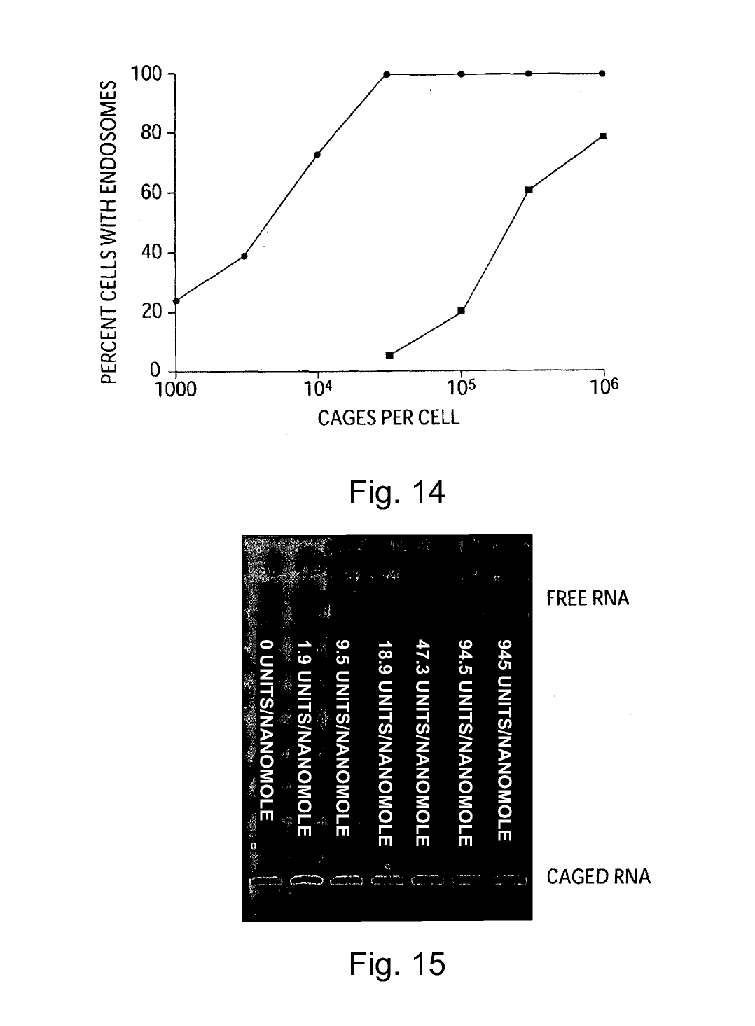

Cage constructs were generated using standard procedures. Following antibody attachment to the delivery system, normal purification of cages away from free antibody using column chromatography was NOT conducted, resulting in the presence of free antibody (>10:1) in targeted cage preparations. Fluorescent internalization experiments were conducted using BCL1 cells and identical experimental conditions as stated above. Experimental incubations for this experiment included the comparison between identical concentrations of targeted cage (purified) and targeted cage (non-purified). Cage concentrations for all experiments are determined by quantifying core protein concentration, so free antibody did not effect concentration calculations. Analysis of internalized delivery system in these experiments was identical to those mentioned above. The results in FIG. 14 show that when targeted nanocages are incubated in the presence of free antibody, a ˜1000 fold decrease internalization is observed. The results in FIG. 14 also show that targeted cages are being internalized through surface marker mediated internalization processes and are not internalized from the local environment thru non specific endocytocic pathways.

### Example 10

The following example describes a Benzonase protection assay. The purpose of this assay is to determine if encapsulation of siRNA molecules with K9 core protein protects it from a range of concentrations of the nuclease, Benzonase.

Free RNA (50 nM) or core-protein encapsulated RNA (150 nM) is injected into 1× Benzonase cleavage buffer with varying amounts of Benzonase (range=1.9 units/nmole to 945 units/nmole) also including a zero benzonase control. This is then incubated for 1 hour at room temperature. The samples are then run on a 1.0% TAE-agarose gel containing ethidium bromide. The gel is then imaged and the intensity of the RNA bands is determined.

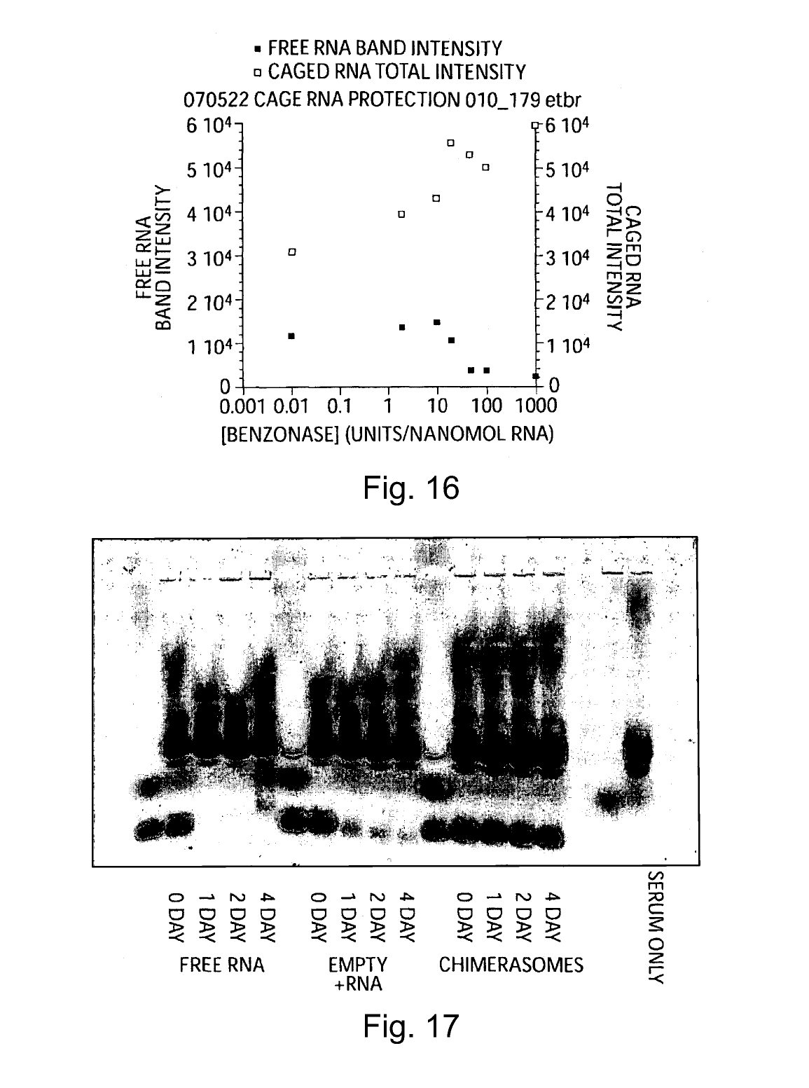

The results in FIG. 15 show that the free RNA band is degraded at about 20 units/nmol, whereas “caged” RNA is does not degrade at any nuclease concentrations tested. This shows that the “caged” RNA is effectively protected. The results in FIG. 16 show the quantification of the bands in FIG. 15. This assay shows that RNA is significantly protected against nuclease activity by encapsulation with K9 core protein

### Example 11

The following example describes a serum protection assay. The purpose of this assay is to determine if the RNA inside of nanocages is protected from serum degradation. Degradation is compared to two control samples: free RNA and Empty nanocages with RNA added after assembly. The second control is to determine whether nanocages protect RNA from serum degradation by some mechanism other than encapsulation.

Mix each RNA preparation as well as serum 1:1 with water as controls. Then mix equal volumes of each RNA sample with human serum, the total sample+serum volume should be between 2 and 4 mL. Freeze several aliquots sample+serum immediately for time zero time points. Then place the remaining samples at 37° C. Remove multiple 50 uL aliquots from samples at regular time points, label and freeze at −80° C. To process the samples add 10% SDS until a final concentration of 0.7% SDS is reached in each sample then incubate at room temperature for 5 minutes. Run the samples at 200 V for 30 minutes on a 1.0% TAE-agarose gel containing ethidium bromide. Quantify intensity of RNA containing bands. Characterize the lifetime of the RNA's to determine the amount of protection.

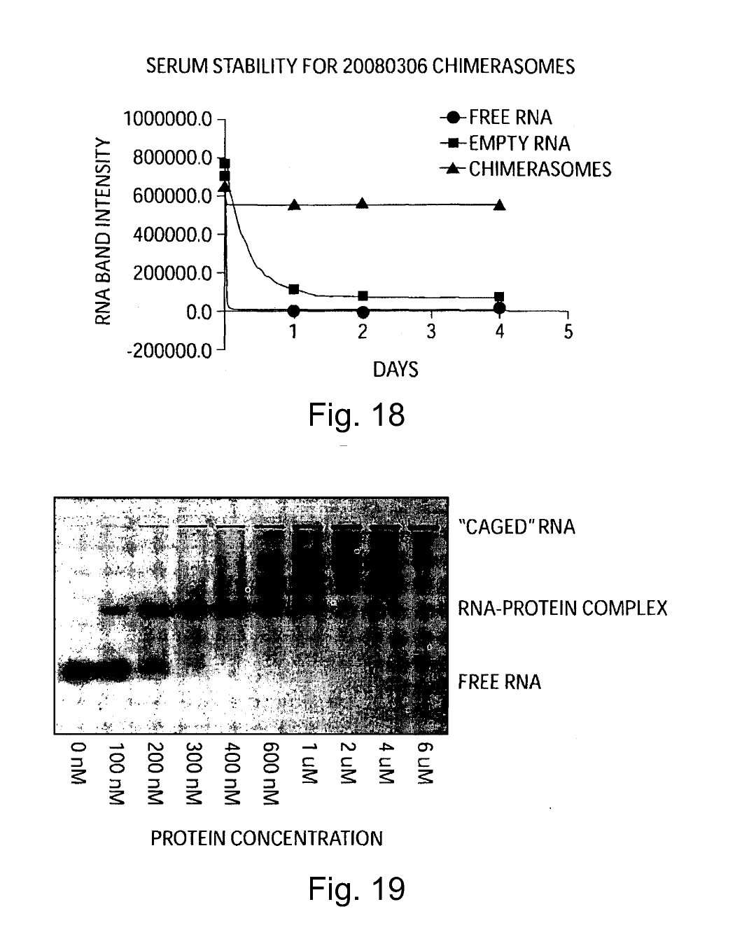

The results in FIG. 17 show that both controls, free RNA and RNA mixed with empty nanocages, had degraded by the first time point (1 day) while the nanocage-protected RNA survived without appreciable degradation for the duration of the experiment, 4 days. The results in FIG. 18 show the quantitation of the RNA bands vs. incubation time for each sample. These results indicated that the nanocage encapsulating RNA protects the RNA cargo from serum degradation at 37° C. Experiments have been run that demonstrate RNA stability is achieved at 14 days without the degradation of the RNA payload. Free RNA, in the presence and absence of empty nanocages, is completely degraded by 1 day.

### Example 12

The following assays determine the K_{d} for K7, K9 and K11 constructs with fluorescent siRNA. The purpose of this study is to determine the affinity of a fluorescent siRNA construct for the HBV core protein mutants, K7, K9 and K11. Below is the sequence of fluorescent siRNA that was used in these experiments.

Siglo Cyclophilin B:

(SEQ ID NO: 31)
DY547-GGAAAGACUGUUCCAAAAAUUUUCCUUUCUGACAAGGUUUUU-P

K9—Siglo Cyclophilin B siRNA K_{d}:

A solution of 20 nM fluorescent duplex (Siglo cycB, RNA from Dharmacon) in 10 mM Tris is labeled f-RNA buffer. K9 protein stock is diluted to 6 μM in f-RNA buffer. The dilution should be performed quickly and on ice, so that nanocage assembly is less apt to form. Make successive dilutions of K9 in f-RNA buffer. Remove RNA-protein dilutions from ice and allow to sit at room temperature for 5 minutes.

A gel is then run of the reactions under the following conditions: Load 15 μL/lane on a 1.5% TAE-agarose gel with duplicate lanes. Run the gel at 200 V for 35 minutes and document gel on a Molecular Dynamics Typhoon scanner. A gel showing free, caged, and protein-bound RNA migrating separately can be seen in FIG. 19.

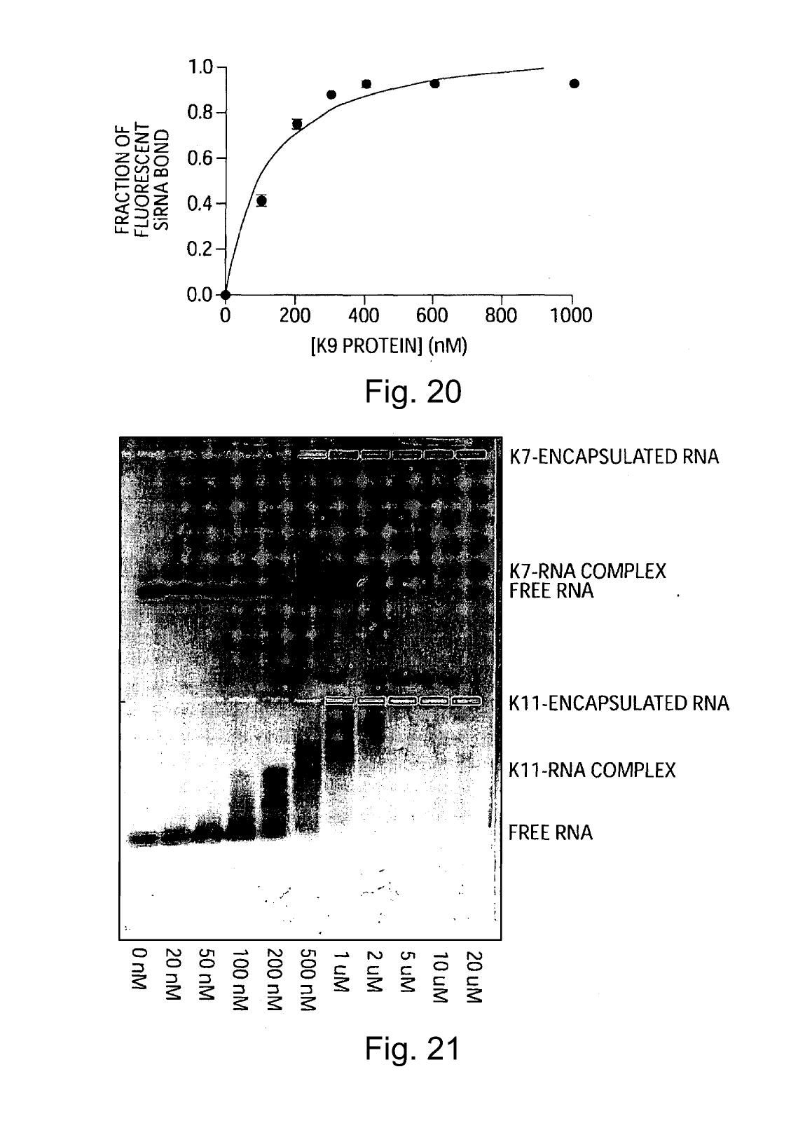

The results in FIG. 20 show that the fluorescent siRNA binds to K9 with a K_{d} of 115 nM. This is a tight affinity, characteristic of RNA-protein interactions. This tight binding affinity is well below the concentrations of RNA and protein used for assembly of nanocages. Therefore, these data suggest the RNA binding sites of K9 protein are saturated with RNA during assembly.

K7 and K11—Siglo Cyclophilin B siRNA K_{d}:

A solution of 20 nM fluorescent duplex (Siglo cycB, RNA from Dharmacon) in 20 mM Sodium Bicarbonate, pH 9.5, is prepared. Both protein stocks were diluted to 40 in the same buffer. FIG. 21 shows that successively diluting the 40 uM protein in 20 mM Sodium Bicarbonate, pH 9.5, generated a range of protein concentrations. RNA and protein solutions were mixed 1:1 and allowed to bind at room temperature for 5 minutes. The final protein concentration in the binding reactions ranged three orders magnitude, from 20 uM to 20 nM. A 1/5 volume of 6×RNA loading buffer (xylene cyanol in 55% glycerol 20 mM Tris, pH 7.7) was added. The samples were then loaded on a 1.0% TAE-agarose gel (13 cm×16 cm), at 80 μL/lane. This was run for 180 V for at 35 minutes and documented gel on a Molecular Dynamics Typhoon scanner. A gel showing free, caged, and protein-bound RNA migrating separately can be seen in FIG. 19.

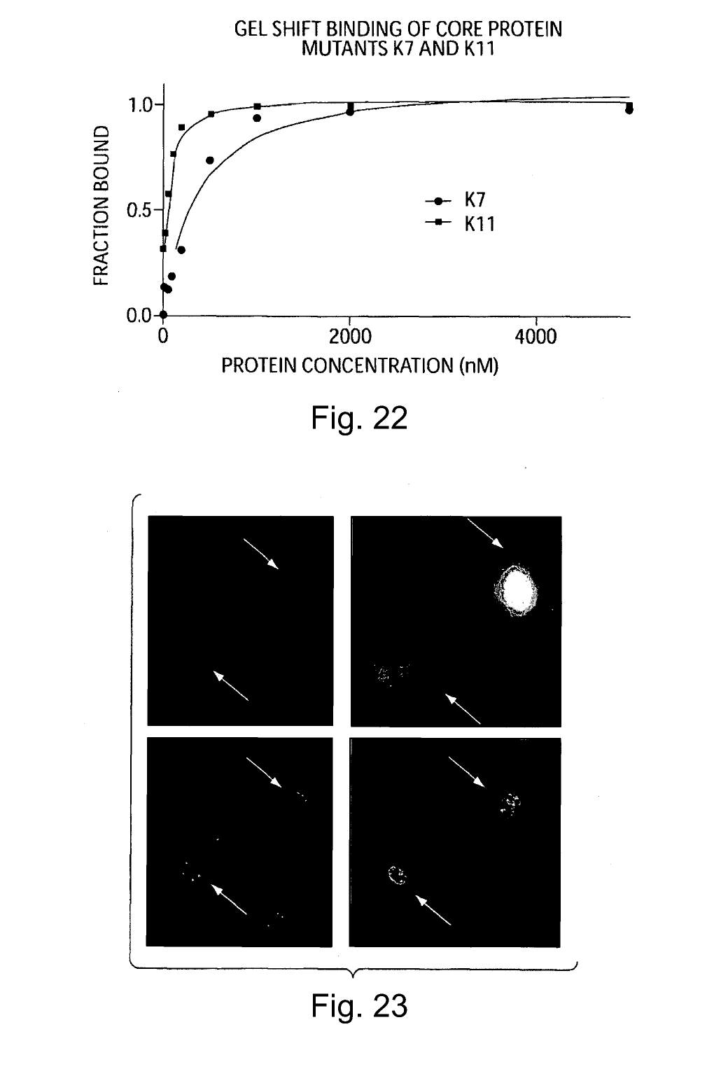

The results in FIG. 22 show that the fluorescent siRNA bound to K7 with a K_{d} of 370 nM and to K11 with a K_{d} of 69 nM. This is a tight affinity, characteristic of RNA-protein interactions. The increase affinity observed for K11 relative to K9 and K7 is attributable to the larger number of cationic residues at the C-terminal end of this protein. As with the mutant K9, the affinity is high enough to fully saturate the RNA binding sites for both mutants during the process of nanocage assembly. Table 2 provides a summary of K7, K9, and K11 mutant binding conditions as well as the K_{d} values.

TABLE 2

| Mutant | Affinity for SiGlo siRNA (K_{d}) | Conditions |
|---|---|---|
| K7 | 370 nM | 20 mM NaHCO3, pH 9.5 |
| K9 | 115 nM | 20 mM Tris, pH 7.7 |
| K11 | 69 nM | 20 mM NaHCO3, pH 9.5 |

### Example 13

The following examples demonstrate the ability of nanocages to encapsulate siRNA, effectively delivery the encapsulated siRNA to a cell and the ability of the encapsulated siRNA to silence or down regulate the activity of a particular gene of interest.

Cell Growth: C166 cells stably expressing the enhanced green fluorescent protein (eGFP) were purchased from ATCC(CRL-2583) and grown at 37° C., 5% CO₂, in DMEM media with 10% fetal bovine serum and supplements (as recommended by ATCC). Cell stocks were grown in T25, T75, or T125 flasks and transferred to 24-well plates for experimentation. Cells were also grown on glass coverslips in 24-well plates when microscopy was to be performed. Cells grown under these conditions consistently exhibited “normal” growth characteristics and doubling times.

Lipid Nanocages Containing Red Fluorescent siRNA Enter Cells

C166 cells were grown on glass coverslips in 24-well plates. Cells were plated onto coverslips 24 hours prior to the addition of the siRNA loaded lipid nanocages. 100 μL of lipid nanocages containing an siRNA directed against Cyclophilin B of SEQ ID NO:31 (3 nM final concentration of siRNA) covalently linked to a red dye in phosphate buffered saline (PBS) were added to 1 mL of media and allowed to sit at 37° C. for 4 hours. Control cells were incubated in 100 μL of PBS (no lipid nanocages present). Cells were then rinsed 3 times in cold PBS and fixed in 1% paraformaldehyde in PBS. Hoescht 33342 (1:10,000) was added for the visualization of cell nuclei, and coverslips were mounted onto glass slides (cells facing down). Slides were then visualized using standard fluorescence microscopy. Microscope settings were held constant for both experimental and control slides. FIG. 23 shows that lipid nanocages containing red fluorescent-labeled siRNA enter C166-eGFP cells when incubated at 3 nM for 4 hours. eGFP expressing C166 cells (eGFP-green and also black and white) stain positively for siRNA-loaded lipid nanocages (red). Cell nuclei are stained in blue.

Lipid Nanocages Containing siRNA Directed Against eGFP Knocks Down eGFP mRNA Expression In Vitro

C166 cells were grown on glass coverslips in 24-well plates. Cells were plated onto coverslips 24 hours prior to the addition of lipid nanocages. 250 μL of lipid nanocages containing an siRNA directed against eGFP (eGFP-19)

(SEQ ID NO: 32)
GCUGACCCUGAAGUUCAUC-dTdT
(SEQID NO: 33)
dTdT-CGACUGGGACUUCAAGUAG

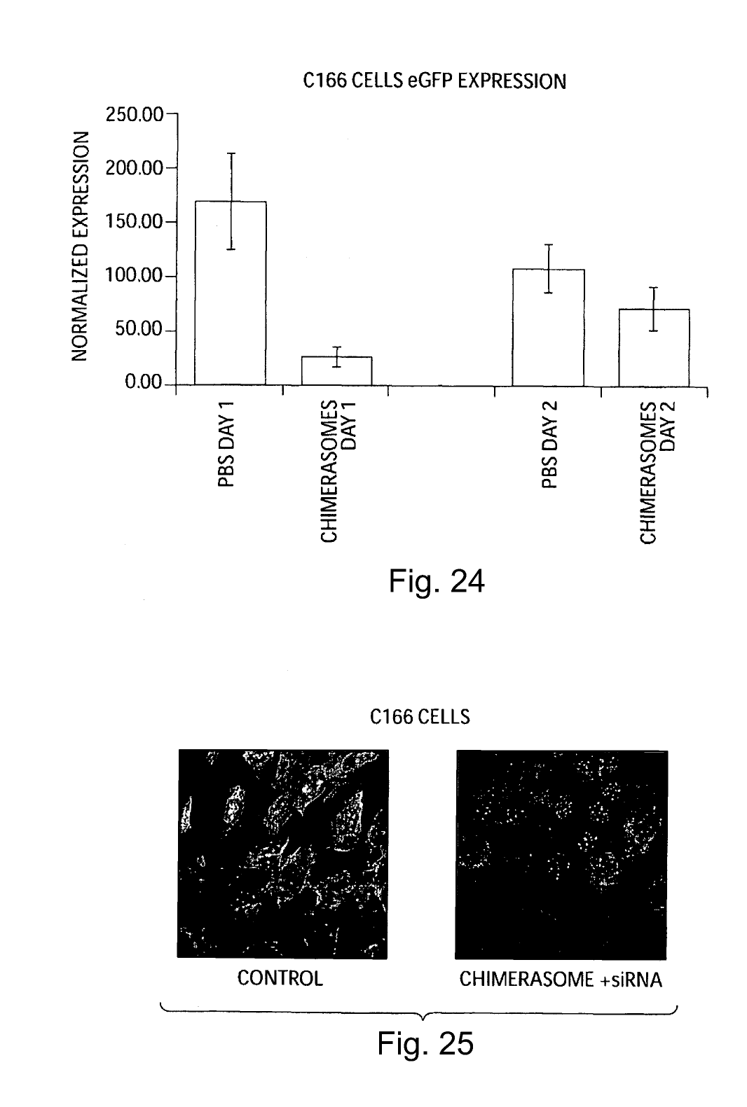

(10 nM final concentration of siRNA) in phosphate buffered saline (PBS) were added to 1 mL of media and allowed to sit at 37° C. for 24 and 48 hours. Control cells were incubated in 250 μL of PBS (no lipid nanocages present). Cells were then rinsed 3 times in cold PBS and homogenized in each well using buffer RLT (Qiagen) with 0.1% BME. Three wells were used for each experimental condition at each time point. RNA was then purified using the RNEasy kit (Qiagen) as recommended by the manufacturer, including an on-column DNAse digestion step. RNA was quantified on the Nanodrop (Thermofisher) and 1 ug of total RNA was reverse transcribed using iScript reverse transcriptase (BioRad) as recommended by the manufacturer. Quantitative polymerase chain reactions (qPCR) were then performed using cDNA, SybrGreen master mix (BioRad) as recommended by the manufacturer, and prequalified primer sets designed using Beacon Designer 6.0 (Premier Biosoft). eGFP gene knockdown was quantified using the ΔΔCt method by comparing eGFP expression levels in each sample to the geometric mean of 3 housekeeping genes in the same sample. All samples were run in triplicate and the average and standard deviations of the three experimental wells were calculated. The results in FIG. 24 show that lipid nanocages containing siRNA directed against eGFP enters cells and knocks down eGFP mRNA expression when incubated at 10 nM for 24 (84% knockdown) and 48 hours (33% knockdown).

Lipid Nanocages Containing siRNA Directed Against eGFP “Knock Down” eGFP Protein Expression In Vitro

C166 cells were grown on glass coverslips in 24-well plates. Cells were plated onto coverslips 24 hours prior to the addition of lipid nanocages. 100 μL of lipid nanocages containing an siRNA directed against eGFP (F-eGFP 19, (SiGlo labeled from Dharmacon)

(SEQ ID NO: 34)
DY547-GCUGACCCUGAAGUUCAUC-dTdT
(SEQ ID NO: 35)
dTdT-CGACUGGGACUUCAAGUAG

(10 nM final concentration of siRNA) covalently linked to a red dye in phosphate buffered saline (PBS) were added to 1 mL of media and allowed to sit at 37° C. for 18 hours. Control cells were incubated in 100 μL of PBS (no lipid nanocages present). Cells were then rinsed 3 times in cold PBS and fixed in 1% paraformaldehyde in PBS. Hoescht 33342 (1:10,000) was added for the visualization of cell nuclei, and coverslips were mounted onto glass slides (cells facing down) in 5% n-propyl gallate in glycerol. Slides were then visualized using confocal microscopy. Microscope gain and PMT settings were held constant for both experimental and control conditions. The results in FIG. 25 show that lipid nanocages containing red fluorescent siRNA directed against eGFP enters cells and knocks down eGFP protein expression after an 18 hour incubation. eGFP (green) expression is reduced in cells incubated with lipid nanocages loaded with siRNA (red). Cell nuclei are labeled blue.

Lipid Nanocages Containing siRNA Directed Against eGFP Knock Down eGFP mRNA Expression In Vivo.

Female C57BL/6-Tg(ACTb-eGFP)1Osb/J mice (˜8 weeks old) received 200 μL tail vein injections of lipid nanocages loaded with a total of ˜620 ng siRNA (eGFP 19 of SEQ ID NO:32 and SEQ ID NO:33) and were sacrificed 24 or 48 hours later. A total of 20 animals received 200 μL of lipid nanocages loaded with siRNA and suspended in PBS, and 20 animals received 200 μL of PBS alone. 16 animals were sacrificed from each group at 24 hours and 4 animals from each group were sacrificed at 48 hours. Liver, kidney, heart, lung, spleen, and pancreas were harvested into RNA later storage solution (Ambion) as recommended by the manufacturer. RNA was purified from ˜25 mg of tissue from each organ using the RNEasy total RNA purification kit and an on column DNAse digestion as recommended by the manufacturer. 1 μg of total RNA was then reverse transcribed using the iScript reverse transcription kit as recommended by the manufacturer. Equal amounts of cDNA were then added to qPCR reactions and levels of eGFP were normalized to the geometric mean of 3 housekeeping genes and percent knockdown was calculated using the ΔΔCt method. All qPCR samples were run in triplicate. Table 3 shows that lipid nanocages containing siRNA directed against eGFP knocks down eGFP mRNA expression in multiple organs in vivo. Percent knockdown in multiple organs was calculated as described above after 24 hours (Day 1) and 48 hours (Day 2). N/A represents no knockdown.

TABLE 3

| Organ | Day 1 (% Knockdown) | Day 2 (% Knockdown) |
|---|---|---|
| Liver | 20 | 68 |
| Kidney | 64 | 14 |
| Heart | 41 | 32 |
| Lung | 25 | 23 |
| Spleen | 22 | 35 |
| Pancreas | N/A | 53 |

Lipid Nanocages Containing siRNA Directed Against eGFP Knocks Down eGFP Protein Expression In Vivo

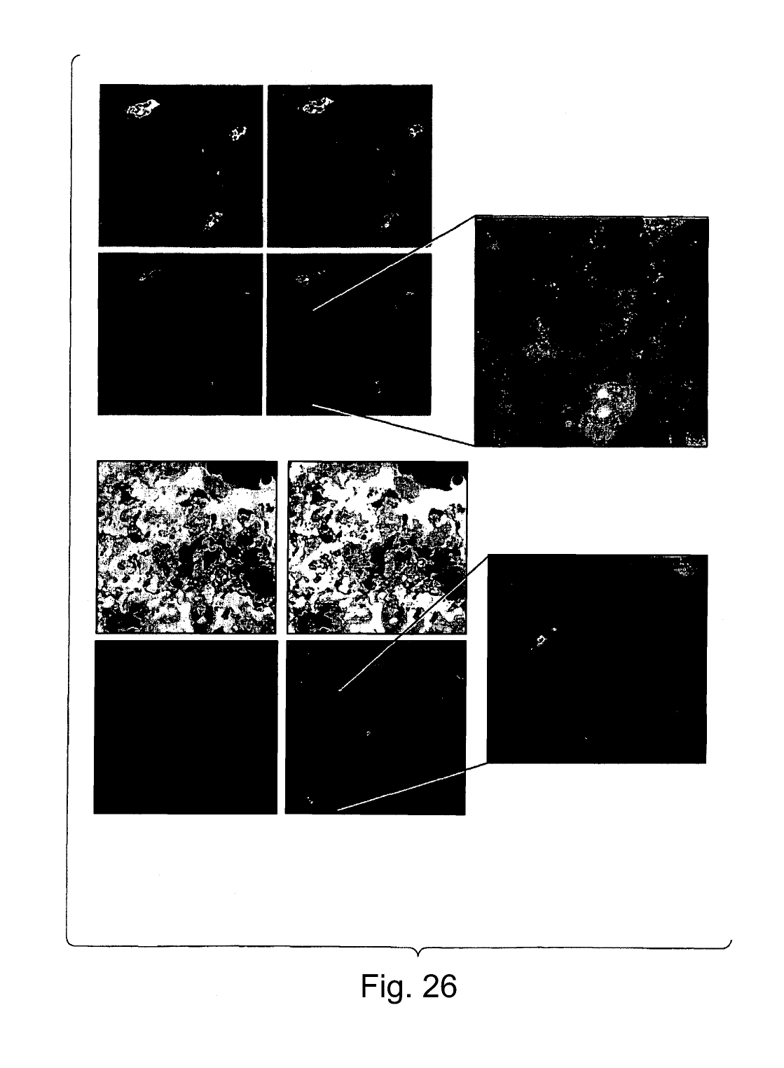

A female C57BL/6-Tg(ACTb-eGFP)1Osb/J mouse (˜9 weeks old) received a 200 μL tail vein injection of lipid nanocages loaded with a total of ˜40 ng siGlo-conjugated siRNA (F-eGFP 19 of SEQ ID NO:34 and SEQ ID NO:35) and was sacrificed 24 hours later. A total of 1 animal received 200 μL lipid nanocages containing siRNA in PBS and 3 naïve animals (female animals from the same litter) received no injection. Liver tissue was harvested and immediately placed in 4% paraformaldehyde in PBS and stored at 4° C. 16 μm frozen sections were cut at −20° C. on a cryostat, coverslipped in 5% n-propyl gallate in glycerol, and viewed using a confocal microscope. All PMT and gain settings were held constant for both experimental and control liver sections. The results in FIG. 26 show that lipid nanocages containing red fluorescent siRNA directed against eGFP knock down eGFP protein expression (green) in the mouse liver in vivo. Green staining represents eGFP and red staining represents fluorescent siRNA (left panels). Black and white images offer better contrast of green and red panels and a high magnification image of the red channel (also black and white) is shown on the right.

Lipid Nanocages Containing siRNA Directed Against eGFP Knock Down eGFP Expression In Vivo

For each mouse liver, from above experiment, 75 μg of tissue was homogenized and extracted in 1.5 mL of PBS-T using a Tissue Lyser (Qiagen). The extract was spun for 10 minutes at 12 g, 4° C. Supernatant was decanted into a fresh 2 mL tube, avoiding transfer of any cloudy liquid at the top. This centrifugation and decanting step was repeated to produce approximately 1 mL of clear liver protein extract. Liver protein extract was stored at −80° C.

Liver extract was diluted 1:1 with PBS and tested for protein concentration with a DC protein assay (BioRad) in a 96-well format. Final calculated protein concentrations were in the range of 2.5 to 3 mg/mL and varied from each other with a standard deviation of 0.2 mg/mL.

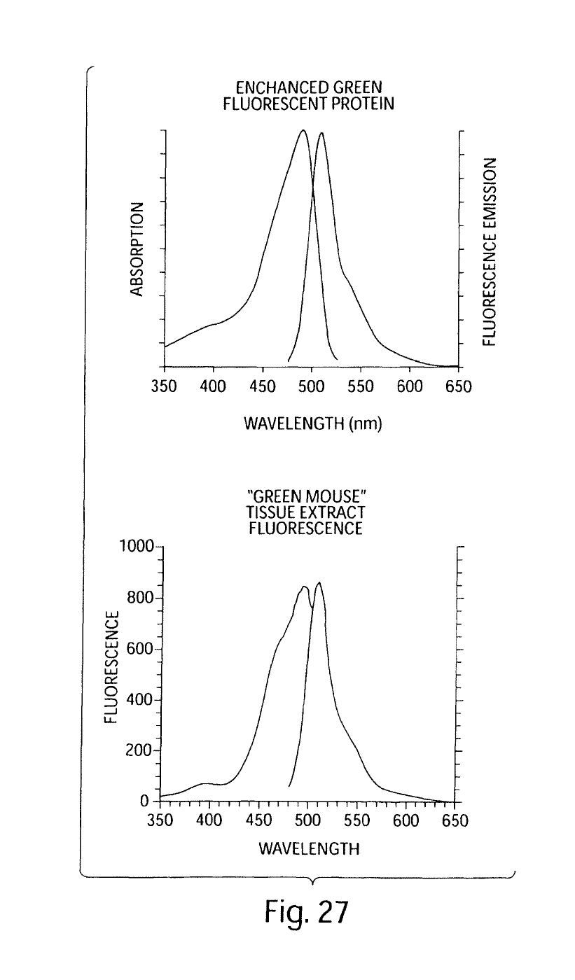

Liver extracts were diluted 1:10 in PBS and tested for EGFP fluorescence on fluorescent spectrophotometer. The results in FIG. 27 show that fluorescent excitation and emission spectra for liver extracts match the corresponding spectra for EGFP. To determine relative levels of EGFP fluorescence from individual liver extracts, 100 μL of 1:10 diluted extract was loaded into wells of a 96 well plate and read on a Turner fluorescent plate reader. Each sample was read in duplicate. For standardization, a standard curve for of 0 to 2 μM fluorescein was also generated from duplicate wells on the same plate.

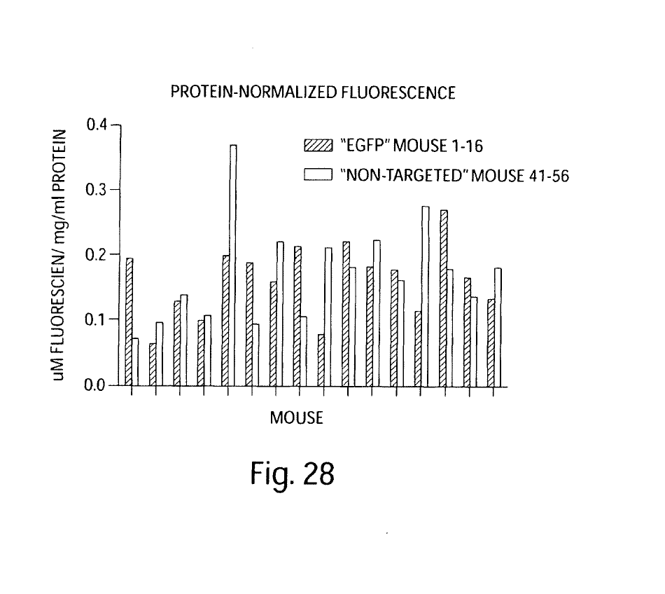

FIG. 28 shows that liver fluorescence values were normalized by the amount of protein and reported as μM Fluorescein equivalents per mg/mL protein. To determine knockdown, samples were averaged and compared for EGFP and non-targeted siRNA treatments. For the 24 hour case, there was a 6% knockdown for the experimental siRNA relative to control with a P-value of 0.34. For the 48-hour case, there was a 36% knockdown for the experimental siRNA relative to control with a P-value of 0.035.

The results are consistent with little EGFP protein knockdown occurring at day 1. At the 48 hour time point, there is significant knockdown of the EGFP protein expression level.

### Example 14

### Anti-CD22 Targeted Nanocages Loaded with Doxorubicin were Evaluated for Their Ability to Target and Kill CD-22 Expressing Cells

B cells (Ramos), and T cells (Jurkat) are added to wells of sterile 96-well plates (500,000 cells/ml) in early log growth phase. Complete growth media (see above) is added to each well after which both CD22-targeted nanocages and non-targeted nanocages loaded with doxorubicin are added across multiple concentrations of nanocage (10 pM, 100 pM, 1 nM, 10 nM, and 100 nM). Cells are assayed for viability using Typan Blue exclusion at multiple time points (12 hr, 24 hr, 36 hr, 48 hr, 60 hr, and 72 hr). Cell viability is normalized to cell viability at the beginning of the experiments for each cell line and is expressed as a % of “normal”. Cell density is also calculated and plotted across each time point for each concentration. All experiments at individual concentrations are conducted in triplicate for each time point.

### Example 15

### Anti-CD22 Targeted Nanocages Loaded with Doxorubicin were Evaluated for their Ability to Reduce Tumor Growth, In Vivo

Female athymic BALB/c nu/nu mice (Harlan Sprague-Dawley), 7-9 weeks of age are maintained according to institutional animal care guidelines on a normal diet ad libitum and under pathogen-free conditions. Five mice are housed per cage. Raji or Ramos cells are harvested in logarithmic growth phase; 2.5-5.0×10⁶ cells are injected subcutaneously into both sides of the abdomen of each mouse. Studies are initiated 3 weeks after implantation, when tumors are 100-300 mm³. Groups consist of untreated, doxorubicin alone, naked nanocages loaded with doxorubicin, and nanocages loaded with doxorubicin and coated with HB22.7.

Tumor volume is calculated by the formula for hemiellipsoids (DeNardo G L, Kukis D L, Shen S, et al., Clin Cancer Res 1997; 3:71-79). Initial tumor volume is defined as the volume on the day prior to treatment. Mean tumor volume is calculated for each group on each day of measurement; tumors that have completely regressed are considered to have a volume of zero. Tumor responses are categorized as follows: C, cure (tumor disappeared and did not regrow by the end of the 84 day study); CR, complete regression (tumor disappeared for at least 7 days, but later regrew); PR, partial regression (tumor volume decreased by 50% or more for at least 7 days, then regrew).

Differences in response among treatment groups are evaluated using the Kruskall Walis rank sum test with the response ordered as none, PR, CR, and Cure. Survival time is also evaluated using the Kruskall Walis test. Tumor volume is compared at 3 time points: month 1 (day 26-29), month 2 (day 55-58), and at the end of the study (day 84). If an animal is sacrificed due to tumor-related causes, the last volume is carried forward and used in the analysis of later time points. Analysis of variance is used to test for differences among treatment groups. P values are two-tailed and represent the nominal p-values. Protection for multiple comparisons is provided by testing only within subsets of groups found to be statistically significantly different.

While the invention has been described in conjunction with the detailed description thereof, the foregoing description is intended to illustrate and not limit the scope of the invention, which is defined by the scope of the appended claims. Other aspects, advantages, and modifications are within the scope of the following claims

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

We claim:

1. A self-assembling nanoparticle drug delivery system comprising:
a capsid comprised of altered, mutated or engineered Hepatitis B Virus (HBV) core proteins,
a bioactive agent captured in said capsid; and
a complex lipid mixture coating said capsid,
wherein the altered, mutated or engineered HBV core proteins are characterized by improved binding affinity of the bioactive agent to the carboxyl terminal portion of the HBV core proteins within the capsid.

2. The self-assembling nanoparticle drug delivery system of claim 1, wherein said mutated or altered HBV core protein has a mutated or altered amino acid sequence of SEQ ID NO:1 or SEQ ID NO:2.

3. The self assembling nanoparticle drug delivery system of claim 2, wherein said HBV core protein comprises a mutation at position 77 such that a glutamic acid is replaced by a cysteine.

4. The self-assembling nanoparticle drug delivery system of claim 3, wherein PE-Malimide is covalently attached to amino acid 77 of the mutated HBV core protein.

5. The self-assembling nanoparticle drug delivery system of claim 2, wherein said HBV core protein comprises an addition of at least six histidine residues to the carboxyl terminus.

6. The self-assembling nanoparticle drug delivery system of claim 2, wherein said HBV core protein further comprises an addition of one to thirty lysine residues to the carboxyl terminus.

7. The self-assembling nanoparticle drug delivery system of claim 6, wherein said HBV core protein further comprises the addition of at least six histidine residues to the carboxyl terminus.

8. The self-assembling nanoparticle drug delivery system of claim 1, wherein said HBV core protein comprises amino acids 1-149 of SEQ ID NO: 1 or 2, wherein the glutamic acid at position 77 is replaced by a cysteine and further comprises the addition of at least five consecutive lysine residues to the carboxyl terminus.

9. The self-assembling nanoparticle drug delivery system of claim 1, wherein said HBV core protein comprises amino acids 1-149 of SEQ ID NO: 1 or 2, wherein the glutamic acid at position 77 is replaced by a cysteine and further comprises the addition of at least six histidine residues to the carboxyl terminus.

10. The self-assembling nanoparticle drug delivery system of claim 1, wherein said HBV core protein comprises amino acids 1-149 of SEQ ID NO: 1 or 2, wherein the glutamic acid at position 77 is replaced by a cysteine and further comprises the addition of at least five consecutive lysine residues and at least six histidine residues to the carboxyl terminus.

11. The self-assembling nanoparticle drug delivery system of claim 1, wherein said HBV core protein comprises the amino acid sequence of SEQ ID NOs: 4, 6, 8, 10, 12, 14 or 16.

12. The self-assembling nanoparticle drug delivery system of claim 2, wherein said HBV core protein comprises a protease recognition site replacing amino acids 79 and 80.

13. The self-assembling nanoparticle drug delivery system of claim 12, wherein said protease recognition site is a thrombin recognition site or a factor Xa recognition site.

14. The self-assembling nanoparticle drug delivery system of claim 2, wherein said HBV core protein is mutated such that at least one amino acid selected from the group consisting of phenylalanine 23, aspartic acid 29, threonine 33, leucine 37, valine 120, valine 124, arginine 127 and tyrosine 132 is changed to a cysteine.

15. A self-assembling nanoparticle drug delivery system of claim 1, wherein said complex lipid mixture comprises at least two lipids selected from the group consisting of cationic, anionic and neutral lipids and further comprises at least one molecule selected from the group consisting of cholesterol, tween, polyethylene glycol and sugars.

16. A self-assembling nanoparticle drug delivery system of claim 1, wherein said complex lipid mixture coats said capsid at a mass value of about 10% to about 60% of the total protein.

17. A self-assembling nanoparticle drug delivery system of claim 6, wherein said complex lipid mixture coats said capsid at a mass value of about 30% of the total protein.

18. A self-assembling nanoparticle drug delivery system of claim 1, wherein said complex lipid mixture comprises 1-Palmitoyl-2-Oleoyl-sn-Glycero-3-[Phospho-rac-(1-glycerol) (POPG), hydrogenated soy phosphatidylcholine (HSPC), and cholesterol.

19. A self-assembling nanoparticle drug delivery system of claim 18, wherein said complex lipid mixture comprises about 60% POPG, about 20% HSPC and about 20% cholesterol.

20. The self-assembling nanoparticle drug delivery system of claim 1, wherein said complex lipid coating further comprises targeting agents selected from the group consisting of lipid conjugated antibodies, peptides, aptamers, ligands or antibody fragments.

21. The self-assembling nanoparticle drug delivery system of claim 20, wherein said antibodies target cellular markers selected from the group consisting of CD 19, CD20, CD22, CD33 or CD74.

22. The self-assembling nanoparticle drug delivery system of claim 1, wherein said bioactive agent is selected from the group consisting of small molecules, proteins, nucleic acids, DNA, RNA, siRNA, miRNA, shRNA, DNA vaccines, peptides, or nucleic acid mimetic molecules.

23. A polypeptide comprising amino acids 1-149 of SEQ ID NO: 1 or 2, wherein the glutamic acid at position 77 is replaced by a cysteine and further comprising the addition of at least five consecutive lysine residues to the carboxyl terminus.

24. A polypeptide comprising amino acids 1-149 of SEQ ID NO: 1 or 2, wherein the glutamic acid at position 77 is replaced by a cysteine and further comprising the addition of at least six histidine residues to the carboxyl terminus.

25. A polypeptide comprising amino acids 1-149 of SEQ ID NO: 1 or 2, wherein the glutamic acid at position 77 is replaced by a cysteine and further comprising the addition of at least five consecutive lysine residues and at least six histidine residues to the carboxyl terminus.

26. The polypeptide of claim 23, wherein said at least five consecutive lysine residues added to the carboxyl terminus increase the polypeptide binding affinity for siRNA to about 50 nm to about 500 nM.

27. The polypeptide of claim 26, wherein said siRNA is about 18 to about 27 nucleotides in length.

28. The polypeptide of claim 25, wherein said at least five consecutive lysine residues added to the carboxyl terminus increase the polypeptide binding affinity for siRNA to about 50 nm to about 200 nM.

29. The polypeptide of claim 28, wherein said siRNA is about 18 to about 27 nucleotides in length.

30. A nucleic acid molecule comprising the nucleic acid sequence of SEQ ID NOs: 3, 5, 7, 9, 11, 13, 15, 36, 38 or 40.

31. A polypeptide comprising the amino acid sequence of SEQ ID NOs: 4, 6, 7, 10, 12, 14, 16, 37, 39 or 41.

32. A method for forming a self-assembling nanoparticle drug delivery system comprising:
(a) mixing a bioactive agent with an HBV core protein modified to have a C-terminal tail with binding affinity of about 10 nM and about 500 nM for the bioactive agent in the presence of a denaturing agent at a concentration of about 1M to about 6M to form a cage solution;
(b) encapsulating said bioactive agent in the core protein cage by raising the ionic strength of said cage solution to obtain a final salt concentration of about 50 mM to about 600 mM and decreasing the denaturing agent concentration to permit assembly of the core protein cage;
(c) adding a lipid linker molecule to facilitate lipid coating of the core protein to said cage solution;
(d) adding a complex lipid coating material comprised of POPG, cholesterol, and HSPC at a mass value of about 10% to about 40% of total protein to said cage solution to form a nanoparticle; and
(e) purifying said nanoparticles.

33. The method for forming a self-assembling nanoparticle drug delivery system of claim 32, wherein said HBV core protein has a mutated or altered amino acid sequence of SEQ ID NO.1 or SEQ ID NO.2.

34. The method for forming a self-assembling nanoparticle drug delivery system of claim 33, wherein said HBV core protein comprises a mutation at position 77 such that a glutamic acid is replaced by a cysteine.

35. The method for forming a self-assembling nanoparticle drug delivery system of claim 33, wherein said HBV core protein comprises an addition of at least six histidine residues to the carboxyl terminus.

36. The method for forming a self-assembling nanoparticle drug delivery system of claim 33, wherein said HBV core protein further comprises an addition of one to thirty lysine residues to the carboxyl terminus.

37. The method for forming a self-assembling nanoparticle drug delivery system of claim 33, wherein said HBV core protein further comprises the addition of at least six histidine residues to the carboxyl terminus.

38. The method for forming a self-assembling nanoparticle drug delivery system of claim 32, wherein said HBV core protein comprises amino acids 1-149 of SEQ ID NO: 1 or 2, wherein the glutamic acid at position 77 is replaced by a cysteine and further comprises the addition of at least five consecutive lysine residues to the carboxyl terminus.

39. The method for forming a self-assembling nanoparticle drug delivery system of claim 32, wherein said HBV core protein comprises amino acids 1-149 of SEQ ID NO: 1 or 2, wherein the glutamic acid at position 77 is replaced by a cysteine and further comprises the addition of at least six histidine residues to the carboxyl terminus.

40. The method for forming a self-assembling nanoparticle drug delivery system of claim 32, wherein said HBV core protein comprises amino acids 1-149 of SEQ ID NO: 1 or 2, wherein the glutamic acid at position 77 is replaced by a cysteine and further comprises the addition of at least five consecutive lysine residues and at least six histidine residues to the carboxyl terminus.

41. The method for forming a self-assembling nanoparticle drug delivery system of claim 32, wherein said HBV core protein comprises the amino acid sequence of SEQ ID NOs: 4, 6, 7, 10, 12, 14, 16, 37, 39 or 41.

42. The method for forming a self-assembling nanoparticle drug delivery system of claim 32, wherein steps (a) and (b) occur under substantially free RNAse conditions.

43. The method for forming a self-assembling nanoparticle drug delivery system of claim 32, wherein said lipid linker molecule of step (c) is PE-Malimide.

44. The method for forming a self-assembling nanoparticle drug delivery system of claim 32, wherein said lipid linker molecule of step (c) is PE-Malimide and wherein said PE-Malimide is covalently attached to amino acid 77 of the mutated or altered amino acid sequence of SEQ ID NO.1 or SEQ ID NO.2.

45. The method for forming a self-assembling nanoparticle drug delivery system of claim 43, wherein PE-Malimide is added at 4 mole equivalents per core protein.

46. The method for forming a self-assembling nanoparticle drug delivery system of claim 32, wherein said complex lipid mixture coats said capsid at a mass value of about 30% of the total protein.

47. The method for forming a self-assembling nanoparticle drug delivery system of claim 32, wherein said complex lipid mixture comprises about 60% POPG, about 20% HSPC and about 20% cholesterol.

48. The method for forming a self-assembling nanoparticle drug delivery system of claim 32, wherein said complex lipid coating further comprises targeting agents selected from the group consisting of lipid conjugated antibodies, peptides, aptamers, ligands or antibody fragments.

49. The method for forming a self-assembling nanoparticle drug delivery system of claim 48, wherein said antibodies target cellular markers selected from the group consisting of CD19, CD20, CD22, CD33 or CD74.

50. The method for forming a self-assembling nanoparticle drug delivery system of claim 32, wherein said bioactive agent is selected from the group consisting of small molecules, proteins, nucleic acids, DNA, RNA, siRNA, miRNA, shRNA, DNA vaccines, peptides, or nucleic acid mimetic molecules.

51. The self-assembling nanoparticle drug delivery system produced by the process of claim 32.

52. A method of regulating gene expression in a cell comprising administering the self-assembling nanoparticle drug delivery system of claim 1, wherein the bioactive molecule is siRNA, wherein the siRNA interferes with the mRNA of the gene to be regulated, thereby regulating expression of said gene.

53. A method of regulating gene expression in a cell comprising administering the self-assembling nanoparticle drug delivery system of claim 51, wherein the bioactive molecule is siRNA, wherein the siRNA interferes with the mRNA of the gene to be regulated, thereby regulating expression of said gene.
